SEQUENCE LISTING (86 sequences)

SEQ ID NO: 1 - DNA, 855 bp, Bacillus macerans
  CDS (1)..(855)
atggcatattcattatggaatgatcaaattgttgaagaaggatctattgcaatctcaccagaagacagaggttatcagtttggtgacggtatttatgaagtaattaaagtttataacggaaatatgtttacagcacaagagcacattgatcgtttctatgcgagcgccgaaaaaattcgccttgttatcccttatacaaaagatgttttacacaagttactacatgagctaattgaaaagaataatctagaaacaggacatgtttattttcaaatcactcgtggggctaattcacgtaatcacgttttcccggatgcaagtattcctgctgtattaactggaaatgtaaaagcgggtgaacgtgcatatgaaaactttgaaaaaggtgttaaagccacttttgttgaggatattcgttggttgcgttgtgacattaaatctttaaacttgcttggtgcagtattagcaaaacaagaagctgcggagaaaggttgttatgaagcgatcttacatcgcggagatatcgtgacagaatgctcttcagctaatgtttacggaattaaagatggaaaactttatacacatccagctaataatttcatcttaaatggtattacacgtcaagtcattttaaaatgtgcggaagaaattaatttaccagtaatcgaagagccaatgacgaaagctgatttactaacaatggatgaaatcattgtgtcgtctgtatcttctgaggttacgccagtcattgatgtggacggcaaccaaattggggctggagttcccggtgaatggactcgtcaattacagcaatcatttgaagcgaaattaccactttcaatgaataccaaataa

SEQ ID NO: 2 - protein, 284 aa, Bacillus macerans
MAYSLWNDQIVEEGSIAISPEDRGYQFGDGIYEVIKVYNGNMFTAQEHIDRFYASAEKIRLVIPYTKDVLHKLLHELIEKNNLETGHVYFQITRGANSRNHVFPDASIPAVLTGNVKAGERAYENFEKGVKATFVEDIRWLRCDIKSLNLLGAVLAKQEAAEKGCYEAILHRGDIVTECSSANVYGIKDGKLYTHPANNFILNGITRQVILKCAEEINLPVIEEPMTKADLLTMDEIIVSSVSSEVTPVIDVDGNQIGAGVPGEWTRQLQQSFEAKLPLSMNTK

SEQ ID NO: 3 - DNA, 38 bp, Artificial Sequence
  Forward primer for preparing D-aminotransferase mutant derived from Bacillus macerans AJ1617 (BmDAT-Nde-f)
ggatgaacggcatatggcatattcattatggaatgatc

SEQ ID NO: 4 - DNA, 30 bp, Artificial Sequence
  Reverse primer for preparing D-aminotransferase mutant derived from Bacillus macerans AJ1617 (BmDAT-delNde-r)
ttcaaagttttcatacgcacgttcacccgc

SEQ ID NO: 5 - DNA, 30 bp, Artificial Sequence
  Forward primer for preparing D-aminotransferase mutant derived from Bacillus macerans AJ1617 (BmDAT-delNde-f)
gcgggtgaacgtgcgtatgaaaactttgaa

SEQ ID NO: 6 - DNA, 46 bp, Artificial Sequence
  Reverse primer for preparing D-aminotransferase mutant derived from Bacillus macerans AJ1617 (BmDAT-Xho-r)
caaggttcttctcgagtttggtattcattgaaagtggtaatttcgc

SEQ ID NO: 7 - DNA, 852 bp, Bacillus proteiformans
  CDS (1)..(852)
atgctctatgtagatgggaaatgggtagaagaagggcaagtcgcagttcatccagaagaccgcggttataactttggggatggcatctatgaggtagtacgcatttataaggggcgcatgtatcaatgggatggacatctaacccgattgtttcgaagtgccaaagaaataaaaatggaccttccatggagcgcagaagagctgacagacttagcgaatcagctgatcaccaagaacaacatcacggagaatgacgacgccagcctttacttgcaagtatctcgtggctctgctccacgtgtacacgatattccctccggaatccagcctgtgataatgggctttgtccgtcgtaaggaccgccctgtcgccgatatgaagaaaggcttgacagctcagctcgttgaagatatccgctggctacgttgcgatatcaaaacactcaacctgctaggtgctgttctagtcaaacaatacgcaaaggatgcaggtgcccaagaatccattctgcaccgcaacggcgtcatcacagagtgcagcgcttctaatttgttcgtcgtaaaaaatggcgagttgtatacgcatcaggctgacaatctgatcctgcacggaattactcgccaagtggtcattgatttggctcggaacaatggcatcactgtccatgaagaagcattcgacattgctttcctaaagcaagccgatgaagtattcctcaccagcacgaccgcggaaatcatgccgctcatctcggtagatggtgtcgcagtcggcaacggacagccaggacctgtcgtgcttacgctacaagacttgtttgaacagcatatcaacacaagtgtactcgtgtaa

SEQ ID NO: 8 - protein, 283 aa, Bacillus proteiformans
MLYVDGKWVEEGQVAVHPEDRGYNFGDGIYEVVRIYKGRMYQWDGHLTRLFRSAKEIKMDLPWSAEELTDLANQLITKNNITENDDASLYLQVSRGSAPRVHDIPSGIQPVIMGFVRRKDRPVADMKKGLTAQLVEDIRWLRCDIKTLNLLGAVLVKQYAKDAGAQESILHRNGVITECSASNLFVVKNGELYTHQADNLILHGITRQVVIDLARNNGITVHEEAFDIAFLKQADEVFLTSTTAEIMPLISVDGVAVGNGQPGPVVLTLQDLFEQHINTSVLV

SEQ ID NO: 9 - DNA, 41 bp, Artificial Sequence
  Primer for preparing D-aminotransferase derived from Bacillus proteiformans AJ3844 (Brevis-F-NdeI)
ggaattccatatgctctatgtagatgggaaatgggtagaag

SEQ ID NO: 10 - DNA, 38 bp, Artificial Sequence
  Primer for preparing D-aminotransferase derived from Bacillus proteiformans AJ3844 (Brevis-F-XhoI)
ccctcgagcacgagtacacttgtgttgatatgctgttc

SEQ ID NO: 11 - DNA, 30 bp, Artificial Sequence
  Forward primer for preparing D-aminotransferase mutant derived from Bacillus macerans AJ1617 (DID-2 H87R)
ctagaaacaggacgtgtttattttcaaatc

SEQ ID NO: 12 - DNA, 30 bp, Artificial Sequence
  Reverse primer for preparing D-aminotransferase mutant derived from Bacillus macerans AJ1617 (DID-2 H87R)
gatttgaaaataaacacgtcctgtttctag

SEQ ID NO: 13 - DNA, 30 bp, Artificial Sequence
  Forward primer for preparing D-aminotransferase mutant derived from Bacillus macerans AJ1617 (DID-8 N100T)
gggctaattcacgtacccacgttttcccgg

SEQ ID NO: 14 - DNA, 30 bp, Artificial Sequence
  Reverse primer for preparing D-aminotransferase mutant derived from Bacillus macerans AJ1617 (DID-8 N100T)
ccgggaaaacgtgggtacgtgaattagccc

SEQ ID NO: 15 - DNA, 35 bp, Artificial Sequence
  Forward primer for preparing D-aminotransferase mutant derived from Bacillus macerans AJ1617 (DID-21 K117R)
gtattaactggaaatgtacgtgcgggtgaacgtgc

SEQ ID NO: 16 - DNA, 35 bp, Artificial Sequence
  Reverse primer for preparing D-aminotransferase mutant derived from Bacillus macerans AJ1617 (DID-21 K117R)
gcacgttcacccgcacgtacatttccagttaatac

SEQ ID NO: 17 - DNA, 35 bp, Artificial Sequence
  Forward primer for preparing D-aminotransferase mutant derived from Bacillus macerans AJ1617 (DID-22 K117Q)
gtattaactggaaatgtacaggcgggtgaacgtgc

SEQ ID NO: 18 - DNA, 35 bp, Artificial Sequence
  Reverse primer for preparing D-aminotransferase mutant derived from Bacillus macerans AJ1617 (DID-22 K117Q)
gcacgttcacccgcctgtacatttccagttaatac

SEQ ID NO: 19 - DNA, 32 bp, Artificial Sequence
  Forward primer for preparing D-aminotransferase mutant derived from Bacillus macerans AJ1617 (DID-23 I145V)
ggttgcgttgtgacgttaaatctttaaacttg

SEQ ID NO: 20 - DNA, 32 bp, Artificial Sequence
  Reverse primer for preparing D-aminotransferase mutant derived from Bacillus macerans AJ1617 (DID-23 I145V)
caagtttaaagatttaacgtcacaacgcaacc

SEQ ID NO: 21 - DNA, 32 bp, Artificial Sequence
  Forward primer for preparing D-aminotransferase mutant derived from Bacillus macerans AJ1617 (DID-27 K157R)
gtgcagtattagcacgtcaagaagctgcggag

SEQ ID NO: 22 - DNA, 32 bp, Artificial Sequence
  Reverse primer for preparing D-aminotransferase mutant derived from Bacillus macerans AJ1617 (DID-27 K157R)
ctccgcagcttcttgacgtgctaatactgcac

SEQ ID NO: 23 - DNA, 32 bp, Artificial Sequence
  Forward primer for preparing D-aminotransferase mutant derived from Bacillus macerans AJ1617 (DID-28 K157Q)
gtgcagtattagcacagcaagaagctgcggag

SEQ ID NO: 24 - DNA, 32 bp, Artificial Sequence
  Reverse primer for preparing D-aminotransferase mutant derived from Bacillus macerans AJ1617 (DID-28 K157Q)
ctccgcagcttcttgctgtgctaatactgcac

SEQ ID NO: 25 - DNA, 32 bp, Artificial Sequence
  Forward primer for preparing D-aminotransferase mutant derived from Bacillus macerans AJ1617 (DID-29 K157T)
gtgcagtattagcaacccaagaagctgcggag

SEQ ID NO: 26 - DNA, 32 bp, Artificial Sequence
  Reverse primer for preparing D-aminotransferase mutant derived from Bacillus macerans AJ1617 (DID-29 K157T)
ctccgcagcttcttgggttgctaatactgcac

SEQ ID NO: 27 - DNA, 33 bp, Artificial Sequence
  Forward primer for preparing D-aminotransferase mutant derived from Bacillus macerans AJ1617 (DID-40 S240T)
gatgaaatcattgtgacctctgtatctaaagag

SEQ ID NO: 28 - DNA, 33 bp, Artificial Sequence
  Reverse primer for preparing D-aminotransferase mutant derived from Bacillus macerans AJ1617 (DID-40 S240T)
ctctttagatacagaggtcacaatgatttcatc

SEQ ID NO: 29 - DNA, 30 bp, Artificial Sequence
  Forward primer for amplifying DNA fragment containing SpAld gene (SpAld-f-NdeI)
ggaattccatatgacccagacgcgcctcaa

SEQ ID NO: 30 - DNA, 30 bp, Artificial Sequence
  Reverse primer for amplifying DNA fragment containing SpAld gene (SpAld-r-HindIII)
gcccaagctttcagtaccccgccagttcgc

SEQ ID NO: 31 - DNA, 29 bp, Artificial Sequence
  Forward primer for converting codon of aldlase gene (6L-f)
acccagacgcgcctgaacggcatcatccg

SEQ ID NO: 32 - DNA, 29 bp, Artificial Sequence
  Reverse primer for converting codon of aldlase gene (6L-r)
cggatgatgccgttcaggcgcgtctgggt

SEQ ID NO: 33 - DNA, 29 bp, Artificial Sequence
  Forward primer for converting codon of aldlase gene (13L-f)
atcatccgcgctctggaagccggcaagcc

SEQ ID NO: 34 - DNA, 29 bp, Artificial Sequence
  Reverse primer for converting codon of aldlase gene (13L-r)
ggcttgccggcttccagagcgcggatgat

SEQ ID NO: 35 - DNA, 29 bp, Artificial Sequence
  Forward primer for converting codon of aldlase gene (18P-f)
gaagccggcaagccggctttcacctgctt

SEQ ID NO: 36 - DNA, 29 bp, Artificial Sequence
  Reverse primer for converting codon of aldlase gene (18P-r)
aagcaggtgaaagccggcttgccggcttc

SEQ ID NO: 37 - DNA, 29 bp, Artificial Sequence
  Forward primer for converting codon of aldlase gene (38P-f)
ctgaccgatgccccgtatgacggcgtggt

SEQ ID NO: 38 - DNA, 29 bp, Artificial Sequence
  Reverse primer for converting codon of aldlase gene (38P-r)
accacgccgtcatacggggcatcggtcag

SEQ ID NO: 39 - DNA, 29 bp, Artificial Sequence
  Forward primer for converting codon of aldlase gene (50P-f)
atggagcacaacccgtacgatgtcgcggc

SEQ ID NO: 40 - DNA, 29 bp, Artificial Sequence
  Reverse primer for converting codon of aldlase gene (50P-r)
gccgcgacatcgtacgggttgtgctccat

SEQ ID NO: 41 - DNA, 42 bp, Artificial Sequence
  Forward primer for converting codons of aldlase gene (77P-81P-84R-f)
cggtcgcgccgtcggtcaccccgatcgcgcgcatcccggcca

SEQ ID NO: 42 - DNA, 42 bp, Artificial Sequence
  Reverse primer for converting codons of aldlase gene (77P-81P-84R-r)
tggccgggatgcgcgcgatcggggtgaccgacggcgcgaccg

SEQ ID NO: 43 - DNA, 873 bp, Artificial Sequence
  Polynucleotide having modified codons, which encodes D-aminotransferase derived from Achromobacter xylosoxidans
catatgatcccgggcgttccgggcgaatcacaagtttatctgaatggcgaatttctgcgtgttgacgaagcaaaagttagcgttctggaccgtggttttattttcggcgatggtatctatgaagtggttccggtttaccagggcaacgcctttcgtatggcagaacatctggatcgtctggaccgtagtctggcagcactgcgtattgcgcaaccgttcgatcgctccggttggattaacctgatccagcaactgctggaacgcaccaatctggatacgtgcatcgtgtatctgcaggttacccgtggtgtcgctaaacgcgaccaccaatttccggcggaaccggtgaagccgacggttttcggcatgattagcgcatgggcaccgccgggtgcagctgtccgtgcccagggtctgagcgcaatttctatcccggatgaacgctggctgcattgtgaaatcaaatcagtttcgctgctgggcaacgtcctggccaagcagcaagcggtggatgcccacgttgacgaagtcctgcagtttcgtgacggcttcctgaccgaaggtagctctacgaatatttgggtcgtgagcggcggtaaactgctggcaccgccgaagaacaatctgattctggaaggtatccgctacggcctgatgggtgaactggcggccgaagcaggcatcgcttttgaagcgcgtcgcctgacccaggatgaagtggcacaagctgacgaactgatgctgagttccgccacgaaagaagtcctgccgattgtgtctctggatggtcgtccggtgggtgcaggtaaaccgggtccggtttatgaacagctgcgtgctggttacgacgcacgtatcgcagctctgctcgag

SEQ ID NO: 44 - protein, 290 aa, Achromobacter xylosoxidans
MIPGVPGESQVYLNGEFLRVDEAKVSVLDRGFIFGDGIYEVVPVYQGNAFRMAEHLDRLDRSLAALRIAQPFDRSGWINLIQQLLERTNLDTCIVYLQVTRGVAKRDHQFPAEPVKPTVFGMISAWAPPGAAVRAQGLSAISIPDERWLHCEIKSVSLLGNVLAKQQAVDAHVDEVLQFRDGFLTEGSSTNIWVVSGGKLLAPPKNNLILEGIRYGLMGELAAEAGIAFEARRLTQDEVAQADELMLSSATKEVLPIVSLDGRPVGAGKPGPVYEQLRAGYDARIAALLE

SEQ ID NO: 45 - DNA, 879 bp, Artificial Sequence
  Polynucleotide having modified codons, which encodes D-aminotransferase derived from Agrobacterium radiobacter
catatgaacatgacctcgccggaacgcaccgtctacgtgaatggccaatactgcccggaaacggaagcaaaagtgagcatcttcgaccgtggttacctgtttgccgatgcaatctacgaagtgacctgcgttctgggcggtaaactggtggatttcgacggtcacatggcgcgtctgcagcgcagcctgaacgaactggatatggccatgccgatgtctgttgacgaactgctggcaattcaccgtaaactggtggcggccaacaatgttgatgaaggcctgatttatatgcagatcagtcgtggttccgcggatcgcgactttaatttcccgccgaagggtaccccgccggtggttgtcatgtttacgcaaagccgtccggtcattgaatctccgctggccaaacgcggcctgaagattatctcactgccggatatccgctggggtcgtcgcgacattaaaaccgtgcaactgctgtatccgtccatggcaaagaacgcagcttaccaggctggcgcggatgacgcttggctggtccaagatggttatgtgacggaagcgagctctgccaatgcatacatcgttcgttcagatggcaaaattgtcacccgcaagctgtcgtttgacattctgcatggtatcacgcgtgcggcagttctgcgtctggcagctgaaagtggctataccctggaagaacgttccttcacgatcgaagaagccctggcggccaaagaagcttttattacctcagcgacgtcgttcgtcaccgcagtgctggaaattgatggcaccacgatcggcgaaggtgttattggtccggtcagcaaacgtctgcgcgaaatttacatcgaaaaagccgtggcagctgcgaagctcgag

SEQ ID NO: 46 - protein, 292 aa, Agrobacterium radiobacter
MNMTSPERTVYVNGQYCPETEAKVSIFDRGYLFADAIYEVTCVLGGKLVDFDGHMARLQRSLNELDMAMPMSVDELLAIHRKLVAANNVDEGLIYMQISRGSADRDFNFPPKGTPPVVVMFTQSRPVIESPLAKRGLKIISLPDIRWGRRDIKTVQLLYPSMAKNAAYQAGADDAWLVQDGYVTEASSANAYIVRSDGKIVTRKLSFDILHGITRAAVLRLAAESGYTLEERSFTIEEALAAKEAFITSATSFVTAVLEIDGTTIGEGVIGPVSKRLREIYIEKAVAAAKLE

SEQ ID NO: 47 - DNA, 852 bp, Artificial Sequence
  Polynucleotide having modified codons, which encodes D-aminotransferase derived from Bacillus megaterium
catatggaactggcatactacgcaggcgaattccgcgacatcaacgaagctgttatcccgatcgacgaacgtggtcatcaatttggtgacggcgtgtatgaatttattcgtgtctacaaaggcgtgccgttctcgatcgaacgccatctggatcgtctggaacgcagcgctaaagcgatttttatcgaactgccggtttctcgtcaggaactgaaggacattatccaccaagccatggaaaaaagcaccctgaaggatgcagacatttatatccaggtcacgcgtggcatcgcaccgcgtaaccacctgtttccggatgtgccggcacagctggcactgaccattcgtcacccgcgccaaacgccggaagaagcgtatgaaaaaggtatctctaccctgctgctggaagacgaacgctgggccaattgctacattaaaagtctgaacctgctgccgaatctgctggcaaagcaggcggccgcatccaaaggctgtaaggaagctattctggtgaaagatggctacatcaccgaaggtagctctagtaacgtgtttgctattaaagacaatgttctgttcaccacgccggcgacccgtaagattctgtcaggtatcacgcgcgccaacgttctggaatgcgctcaggaacaagcgattcagatccgtgaacaaaatatgaccccgtcatttctgaaagatgccgacgaagttttcattacctccacgtcagttggcctgctgccggtctcgaaaatcgatgaagtcgaactgccgacctttcgtccggtgacggttgccctgaaacatgcatattccctgcgcaccgctggtacgaaggcgtaccagctcgag

SEQ ID NO: 48 - protein, 283 aa, Bacillus megaterium
MELAYYAGEFRDINEAVIPIDERGHQFGDGVYEFIRVYKGVPFSIERHLDRLERSAKAIFIELPVSRQELKDIIHQAMEKSTLKDADIYIQVTRGIAPRNHLFPDVPAQLALTIRHPRQTPEEAYEKGISTLLLEDERWANCYIKSLNLLPNLLAKQAAASKGCKEAILVKDGYITEGSSSNVFAIKDNVLFTTPATRKILSGITRANVLECAQEQAIQIREQNMTPSFLKDADEVFITSTSVGLLPVSKIDEVELPTFRPVTVALKHAYSLRTAGTKAYQLE

SEQ ID NO: 49 - DNA, 858 bp, Artificial Sequence
  Polynucleotide having modified codons, which encodes D-aminotransferase derived from Bacillus halodurans
catatggactattgcctgtatcaagatcaactggtgccgcgtgaacaactgaagattgacccggaagaccgtggctatcattttggcgatggcatttatgaagtggttcatgtgtaccacggcaaagcgtttgccctgtccgatcatctgacgcgtttcaaagaatcagcagaaaagctggatctgccgatgctgtattctaccgacaaactgggtgaactggttcagcaactgattgaaaagaacaagctggaacatggcatggtctattttcagatgacccgtggtatctccccgcgcaaccacctgtacacccgcaatgaaacgccggtgctgaccggcttctcgaaaccgctgccggatgaaaagcgtgaaagcgttcgcctgtacctgacggatgacattcgttggctgcgctgcgacattaaaaccatcaacctgctgggtaatgtcctggcgaagcgtgaagccaccgatcatcagtgtgacgaagcactgctgcaccgtgatggtaccgtcacggaaggtagctctagtaacgtgtttctgatcaaaaatgaaaccctgtatacgcatccggccacgaacctgattctgaatggcatcacgcgtcaaattaccatccgcctggcaaaagctaagggttacaccgtcgtggaagaaccgtttccgaaagaagttattaaggatgcagacgaagctttcatcaccagcacgattcatgaaatcaccccggttacggaagtcattggcgatgaaacggcccactttccggtgggtccggttaccaaaatgctgcagcaagcgttcgccgaagaaatcgcaaaacacagtcagaccgctatgaagcaactcgag

SEQ ID NO: 50 - protein, 285 aa, Bacilllus halodurans
MDYCLYQDQLVPREQLKIDPEDRGYHFGDGIYEVVHVYHGKAFALSDHLTRFKESAEKLDLPMLYSTDKLGELVQQLIEKNKLEHGMVYFQMTRGISPRNHLYTRNETPVLTGFSKPLPDEKRESVRLYLTDDIRWLRCDIKTINLLGNVLAKREATDHQCDEALLHRDGTVTEGSSSNVFLIKNETLYTHPATNLILNGITRQITIRLAKAKGYTVVEEPFPKEVIKDADEAFITSTIHEITPVTEVIGDETAHFPVGPVTKMLQQAFAEEIAKHSQTAMKQLE

SEQ ID NO: 51 - DNA, 849 bp, Artificial Sequence
  Polynucleotide having modified codons, which encodes D-aminotransferase derived from Coprococcus comes
catatgaagaccctgggttactacaatggcaagtttggcccgctggaagaaatgaccgtcccgatgaatgaccgtgcgtgctactttggtgacggcgtgtatgaagcaaccctggctcgtaacggtaaaatctttgcactgaaggaacatctggatcgctttttcaatagcgctggtctgattaaaatcgatattccgtataccaaggacgaactggcggccattctgtacgatatgctggccaaaatggatgacaaggacatcttcatttactggcagatgacccgtggcacgggtatccgccagcatcaatttccggcagctggcaccaaaccgaacctgtggattttcatgaaaccgggcaagtctgcagatagctctaaacgtctgaagctgaccgatatggaagacacgcgctttctgcactgcaatatcaaaaccctgaacctgctggtgaatgttatggcggccgaaaaggctgaatccctgggctgcggtgaatgtgtctttcatcgtggtgatattgtgacggaatgcgcgcactcgaacgttagcattatcaaagatggcgtcttcaagacccatccggccgaccactatatcctgccgggtattacgcgcatgcacatcatccagatctgtaaggataacggcatcatcgtcgacgaaaccccgtttacgatggcggaactgatggatgccgacgaaattatcgtgagttcctcaaccaaattctgcagtccggtttgtgaaatgaacggtacgccgatcggcggtaaagcaccggaactggttgatctgatccagaaaaaatacaccgaaaagttcgaaaacgaaacgatgctcgag

SEQ ID NO: 52 - protein, 282 aa, Coprococcus comes
MKTLGYYNGKFGPLEEMTVPMNDRACYFGDGVYEATLARNGKIFALKEHLDRFFNSAGLIKIDIPYTKDELAAILYDMLAKMDDKDIFIYWQMTRGTGIRQHQFPAAGTKPNLWIFMKPGKSADSSKRLKLTDMEDTRFLHCNIKTLNLLVNVMAAEKAESLGCGECVFHRGDIVTECAHSNVSIIKDGVFKTHPADHYILPGITRMHIIQICKDNGIIVDETPFTMAELMDADEIIVSSSTKFCSPVCEMNGTPIGGKAPELVDLIQKKYTEKFENETMLE

SEQ ID NO: 53 - DNA, 873 bp, Artificial Sequence
  Polynucleotide having modified codons, which encodes D-aminotransferase derived from Geobacillus sp.
catatgaccctgaaactgtatgtcctgaccgaaaagcaattcctgccgcgccatgaagtgacctatccgatggaagaacgtggtatgcaatttggcgacggtgtctatgaagtggcccgtatttatcagggtacctactttctgctggaagaacatattgatcgtctgtaccgttcggcagcagcaatccgtctgagcgtcccgttcgaaaaagacgtgctgatggaaaagctggaactgctgcgcgaaatgaacaatgtgaaagaagatgctattctgtatctgcaggttacgcgtggttcttttccgcgcaaccacgctttcccggcggaaaaccgcccgaatctgtatgcctacatccgtgaaatgccgcgcaaaatgcaagaaattgaaaatggcgtccgtaccatcctgacgaaggatgttcgctgggaatattgctacattaaaagcctgaacctgctgccgaatgtgctggctaagcaggaagcggttgaacgtcaagcctttgaagcaattctgcatcgcgatggcattatcaccgaaggtagctctagtaacatcttcctggtgaaagacggcaatgtttatacccacccggcaacggaacgtattctgaacggtatcgttcgcatgaaagtcaagcagttttgttctgaactgggcattccgctgatcgaagaagcgtttagtattaatgatatcgccgaagcagacgaaatgttcctgacctccaccacgtcctcaattatcccgatcacgcaggtggaagaacaagtggttggcgatggtaaaccgggcgaagttacccgtaagctgcaagtcgcctacgaaaaagctgcgggcctggcagtgaaaagcggtaagctcgag

SEQ ID NO: 54 - protein, 290 aa, Geobacillus sp.
MTLKLYVLTEKQFLPRHEVTYPMEERGMQFGDGVYEVARIYQGTYFLLEEHIDRLYRSAAAIRLSVPFEKDVLMEKLELLREMNNVKEDAILYLQVTRGSFPRNHAFPAENRPNLYAYIREMPRKMQEIENGVRTILTKDVRWEYCYIKSLNLLPNVLAKQEAVERQAFEAILHRDGIITEGSSSNIFLVKDGNVYTHPATERILNGIVRMKVKQFCSELGIPLIEEAFSINDIAEADEMFLTSTTSSIIPITQVEEQVVGDGKPGEVTRKLQVAYEKAAGLAVKSGKLE

SEQ ID NO: 55 - DNA, 873 bp, Artificial Sequence
  Polynucleotide having modified codons, which encodes D-aminotransferase derived from Geobacillus toebii
catatgaccctgaaactgtatgtgctgaccgaaaaacaattcctgccgcgccatgaagtgacctatccgatggaagaacgtggcctgcaatttggcgatggtgtgtatgaagttgcccgtatttatcagggcacctacttcctgctggaagaacatattgaccgtctgtaccgttcggcagcagcaatccgtctgagcgttccgtttgataaagacgtcctgatggaaaagctggaactgctgcgcgaaatgaacaatgtcaaagaagatgccattctgtatctgcaagtgacccgtggcagcttcccgcgtaaccacgcctttccggcagaaaaccgtccgaatctgtatgcatacatccgcgaaatgccgcgtaaaattcgcgaaatcgaaaatggtgtgcgcaccattctgacgcgtgatgttcgctgggaatattgctacatcaagagtctgaacctgctgccgaatgtgctggccaaacaggaagcaaccgaacgtggcgcttttgaagcgattttctatcgcgatggcgacatcacggaaggtagctctagtaacattttcctggtcaaagatggcaaggtgtacacccatccggctacggaacgtatcctgaatggtattatccgcatgaaagttaaggaattttgtgacctgttccacattccgtttgtcgaagaagcattctctattgaagatatcgctcaggcggacgaaatgtttctgacctccaccacgtcctcaattatcccgattatccaggtggaagaacaactgatcgcggatggcaaaccgggtgaagttacgcgtaagctgcaagctgcgtatgaaaaagccgcaggtctggctgttaaaaacgcgaagctcgag

SEQ ID NO: 56 - protein, 290 aa, Geobacillus toebii
MTLKLYVLTEKQFLPRHEVTYPMEERGLQFGDGVYEVARIYQGTYFLLEEHIDRLYRSAAAIRLSVPFDKDVLMEKLELLREMNNVKEDAILYLQVTRGSFPRNHAFPAENRPNLYAYIREMPRKIREIENGVRTILTRDVRWEYCYIKSLNLLPNVLAKQEATERGAFEAIFYRDGDITEGSSSNIFLVKDGKVYTHPATERILNGIIRMKVKEFCDLFHIPFVEEAFSIEDIAQADEMFLTSTTSSIIPIIQVEEQLIADGKPGEVTRKLQAAYEKAAGLAVKNAKLE

SEQ ID NO: 57 - DNA, 867 bp, Artificial Sequence
  Polynucleotide having modified codons, which encodes D-aminotransferase derived from ID220 disclosed in WO2009/088482A1
catatggacgctctgggttattacaatggcaactggggtccgctggatgaaatgacggttccgatgaatgatcgtggctgttactttggtgacggcgtgtatgatgcgacctgcgccgtcaacggtgtgatctttgcgctggatgaacatattgaccgtttctttaactctgccaaactgctggaaatcaatattagtctgaccaaggaagaactgaaaaagacgctgaacgaaatgtactctaaggtggataagggcgaatatctggtttactggcaggtcacccgcggcacgggtcgtcgctcgcatgtttttccggcgggtccgagcaacctgtggattatcattaaaccgaaccacatcgataatctgtaccgtaaaatcaagctgattaccatggatgacacgcgcttcctgcactgcaatattaaaaccctgaacctgatcccgaatgtcattgcatcccagcgtgcactggaagctggctgccatgaagctgtctttcaccgcggtgaaaccgtgacggaatgtgcgcattcaaacgttcacatcattaaaaatggccgtttcatcacccatccggctgataacctgattctgcgtggtacggcacgctcacacctgctgcaagcttgtgtgcgtctgaatatcccggttgacgaacgcgaattttccctgtcagaactgttcgatgccgacgaagtgctggttagctctagtggcaccctgggtctgtcggcagaagaaattgatggcaaaaaggcgggcggtaaagccccggaactgctgaaaaagatccaagacgaagttctgcgtgaattcattgaagccaccggttacacgccggaatggagccgcgtcctcgag

SEQ ID NO: 58 - protein, 288 aa, Unknown
  Obtained from environmental sample (ID220 disclosed in WO2009/088482A1)
MDALGYYNGNWGPLDEMTVPMNDRGCYFGDGVYDATCAVNGVIFALDEHIDRFFNSAKLLEINISLTKEELKKTLNEMYSKVDKGEYLVYWQVTRGTGRRSHVFPAGPSNLWIIIKPNHIDNLYRKIKLITMDDTRFLHCNIKTLNLIPNVIASQRALEAGCHEAVFHRGETVTECAHSNVHIIKNGRFITHPADNLILRGTARSHLLQACVRLNIPVDEREFSLSELFDADEVLVSSSGTLGLSAEEIDGKKAGGKAPELLKKIQDEVLREFIEATGYTPEWSRVLE

SEQ ID NO: 59 - DNA, 864 bp, Artificial Sequence
  Polynucleotide having modified codons, which encodes D-aminotransferase derived from Halothiobacillus neapolitanus
catatgcaagcggtctattttaatggtcgttggattgctccggaagaagcgaacatctcggcgtttgatcgtggttttctgtttggtgacggcgtctatgaagtgattccggcctttaaccgtcgcctgttcggtgcaggtgcacatctggaccgtctgacccgcagtctggatcagattgacatccaagatccgctgacgcgtgctcagtggatggatgtgctggttcgcctggtttcagaatgcggcgcggatgacgtctcgatttatatccaggtgacccgtggtgcaacggctaaacgcgatcacgcttacccgaatccgccgctgccgccgaccgttctggcaagtgcctccgcaattgtcccgctgagcgcggaaatctttaccaaaggcgctaaggcaatcacggtgccggacctgcgttggggtcgttgtgatattaaaagcgtgaacctgctgccgaatatcatggctcgtcagcaagcagtggcagcaggtgccgttgaagcaattatggtccgcgaaggcatcgccctggaaggtgcagcttccaacctgttcgccgttattgatgacgaactgctgaccgcaccgctgggtccgcatatcctgggcggtgtgacgcgtaatcgcctggttgacatggtcaaagatcagggtcaaattccgctgctggaagtcccgatcccgtttgaccgtctgttcgatgcaaccgaagtgtttatgaccagctctacgcgtgatctgctgccgattacccgtatcaacgcacacccggtgggtacgggcaaggttggtccgatttggaccaaactgagcaaggcattcacgaaactgaagcagcaacgcgaaaccctcgag

SEQ ID NO: 60 - protein, 287 aa, Halothiobacillus neapolitanus
MQAVYFNGRWIAPEEANISAFDRGFLFGDGVYEVIPAFNRRLFGAGAHLDRLTRSLDQIDIQDPLTRAQWMDVLVRLVSECGADDVSIYIQVTRGATAKRDHAYPNPPLPPTVLASASAIVPLSAEIFTKGAKAITVPDLRWGRCDIKSVNLLPNIMARQQAVAAGAVEAIMVREGIALEGAASNLFAVIDDELLTAPLGPHILGGVTRNRLVDMVKDQGQIPLLEVPIPFDRLFDATEVFMTSSTRDLLPITRINAHPVGTGKVGPIWTKLSKAFTKLKQQRETLE

SEQ ID NO: 61 - DNA, 855 bp, Artificial
  Polynucleotide having modified codons, which encodes D-aminotransferase derived from ID896 disclosed in WO2009/088482A1
catatgaaagaactgggctactataatggtaaaattggcgaactgtccgaaatgaccgtcccgatgaacgaccgtgcgtgttggtttggtgacggcgtgtatgaagcaggcatgtgccgtgatggccagatttttgctctggatgaacatgttgaccgtctgttccgctcggcggccatgctggaaattcgcgtgccggttagcaaagaagaactgaagcagctgctgtacgatctgatcggtaaaatggataccggcgacctgctggtctattaccaagtgacgcgtggctgcggtccgcgcggtcatgcatttccggatggtccggcctctctgtgggttaccctgcgtccgaagacgctgaacccgttcctgccgcctgtcagtctgattaccgatccggacacgcgctttttccactgtaacattaaaaccctgaatctgatcccgtcagtgatggcaaacgaaaaggcgcgtcgcgccggctgctatgaatgtgtcctgtaccgtccgggcggtcgtgtgaccgaatgctcccattcaaatgttcacattatccgtgatggtgtctttatcaccgccccgacggacgaactgattctgccgggcatcgcacgtgctcatctgattcgcgcatgtagcgctctgggtatcccggtgcgcgaaaccccgttcacggttgaagatctgaaagcagctgacgaaattctgaccagctctagtacggcaccgtgcatccgtgcttgtcgtgtcgatggtcaggcagccggtatgcgtcgcccggacctgtttgacgcgctgcaccgtgccgtttttgacgaatatttcggcaatcaaggttggcgctccctcgag

SEQ ID NO: 62 - protein, 284 aa, Unknown
  Obtained from environmental sample (ID896 disclosed in WO2009/088482A1)
MKELGYYNGKIGELSEMTVPMNDRACWFGDGVYEAGMCRDGQIFALDEHVDRLFRSAAMLEIRVPVSKEELKQLLYDLIGKMDTGDLLVYYQVTRGCGPRGHAFPDGPASLWVTLRPKTLNPFLPPVSLITDPDTRFFHCNIKTLNLIPSVMANEKARRAGCYECVLYRPGGRVTECSHSNVHIIRDGVFITAPTDELILPGIARAHLIRACSALGIPVRETPFTVEDLKAADEILTSSSTAPCIRACRVDGQAAGMRRPDLFDALHRAVFDEYFGNQGWRSLE

SEQ ID NO: 63 - DNA, 867 bp, Artificial Sequence
  Polynucleotide having modified codons, which encodes D-aminotransferase derived from ID892 disclosed in WO2009/088482A1
catatggatgctctgggctactacaatggtaaatggggtccgctggatgaaatgaccgttccgatgaatgaccgtggctgctactttggtgacggcgtctatgatgcaaccatcgcggccaacggtgtgatttttgctctggatgaacatattgaccgtttctttaatagctctaaactgctggaaattaagatctgcattaccaaagaagaactgaaaaagacgctgaacgatatgcactttaaagtggacaagggcgtttatatggtctactggcaggctacccgtggcacgggtcgtcgcaaccatgttttcccggccggtccgagcaatctgtggatcatgattaagccgaaccacatcgatgacctgaacaaaaagatcaagctgatcaccacggaagatacccgcttcctgcattgcaatattaaaacgctgaacctgatcccgaatgtgattgcatctcagcgtgcactggaagctggctgccaagaagctgtgtttcaccgcggtgaaaccgttacggaatgtgcgcattccaacgttcacattatcaaaaatggccgcttcatcacccatcaggcggataacctgatcctgcgtggtattgcgcgcagtcacctgctgcaagcctgtgatcgtctgaatgtgccggttgacgaacgcgaatttaccctgccggaactgttcgatgcggacgaagtcctggtgagttcctcaggcacctttggtctgtcggccgatacgattgacggcaagagcgttggcggtaaagcaccggaactgctgaaaaagatccaagatgaagtcatgcgtgaattcattgaagcgaccggttacacgccggaatggcgcaaagccctcgag

SEQ ID NO: 64 - protein, 288 aa, Unknown
  Obtained from environmental sample (ID892 disclosed in WO2009/088482A1)
MDALGYYNGKWGPLDEMTVPMNDRGCYFGDGVYDATIAANGVIFALDEHIDRFFNSSKLLEIKICITKEELKKTLNDMHFKVDKGVYMVYWQATRGTGRRNHVFPAGPSNLWIMIKPNHIDDLNKKIKLITTEDTRFLHCNIKTLNLIPNVIASQRALEAGCQEAVFHRGETVTECAHSNVHIIKNGRFITHQADNLILRGIARSHLLQACDRLNVPVDEREFTLPELFDADEVLVSSSGTFGLSADTIDGKSVGGKAPELLKKIQDEVMREFIEATGYTPEWRKALE

SEQ ID NO: 65 - DNA, 1389 bp, Artificial Sequence
  Polynucleotide having modified codons, which encodes D-aminotransferase derived from ID904 disclosed in WO2009/088482A1
catatgccggacaccctgaccaccaactcaccgattattagtgcttaccgtgctgcgacgccgggctctgccgaacatgctcaacgtgctgctgaaatgtttccgagcggtattacccatgattcccgctatatcgaaccgtatggcatttacatcgcacgcgctcagggtccgcgtaaatgggatgttgacggccgttgctatgtcgactacttcggtggtcatggtgcactgctgctgggtcattgtcacccggaagtcatggcagcagtgcacgcacaactggatcgtggcacccattatggtgccagtcacgaactggaaattgaatgggccgaacgtgttaaagcactgatcccgaccgctgaacgcgtccgttttacgagctctggtaccgaagccacgctgatggcagttcgtctggcgcgtgccttcaccggtaaaccgaagctgattcgctttaactaccatttccacggttggcatgatcacatgaccagcggccatgcgaatcactttgacggcacgccgaccacgggtgttctggatgcagtcgctggcaacgtgctgctgtgcgatcagaatgacgaagcagctctggcccgcctgctggatcgtcatcacggtgaaattgcggccgcaattatcgaaccgaccggtgcaaacggtggtaaactgccgatcgatccggacttcctgcaggcactgcgtcgcctgacgtctgaacatggtgtgctgctgatctttgacgaagtggttaatggtttccgtgttgcaccgggcggtgctcaagaagcgtacggtattcgtccggatctgaccacgctggctaagatcctggcgggcggtctgccgggcggtgcagtcaccggccgcaaagatattctggacctgctggattttcaagtgaccaagggcgccggtaaagaaaagatcaaccatccgggcacgttcaacgcgaatccgctgtcagctgcggccggcattgcagctctgaaaatcgttcgtgaatcggacgcatgcgcccgtgcaaaccactatggtgatgaactgcgtcgccgtctgaatgaagtgtttgaagaagaacgtatgccgtgggcggcctacggtacctttagcacgctggaactgttcaccaacccggaaggcaaaaagatttcaccgtcgacgtttgatccgctggaagaaagcttcgcgtctctgaaaggcgaacgcaatgccggtattatccataagctgcgtctgggcatgatgattcatggtgtcgacctgagttcccacccgggcggtgtgatcagctgtacccacggtgaagcagaaatggaagataccgtgaaagctatgcgttctacggttcgcatgctgcgtgcggaaggcgaactgctcgag

SEQ ID NO: 66 - protein, 462 aa, Unknown
  Obtained from environmental sample (ID904 disclosed in WO2009/088482A1)
MPDTLTTNSPIISAYRAATPGSAEHAQRAAEMFPSGITHDSRYIEPYGIYIARAQGPRKWDVDGRCYVDYFGGHGALLLGHCHPEVMAAVHAQLDRGTHYGASHELEIEWAERVKALIPTAERVRFTSSGTEATLMAVRLARAFTGKPKLIRFNYHFHGWHDHMTSGHANHFDGTPTTGVLDAVAGNVLLCDQNDEAALARLLDRHHGEIAAAIIEPTGANGGKLPIDPDFLQALRRLTSEHGVLLIFDEVVNGFRVAPGGAQEAYGIRPDLTTLAKILAGGLPGGAVTGRKDILDLLDFQVTKGAGKEKINHPGTFNANPLSAAAGIAALKIVRESDACARANHYGDELRRRLNEVFEEERMPWAAYGTFSTLELFTNPEGKKISPSTFDPLEESFASLKGERNAGIIHKLRLGMMIHGVDLSSHPGGVISCTHGEAEMEDTVKAMRSTVRMLRAEGELLE

SEQ ID NO: 67 - DNA, 855 bp, Artificial Sequence
  Polynucleotide having modified codons, which encodes D-aminotransferase derived from Paenibacillus larvae
catatggaagcatccccgatcctgtatctgtataataatcacattgtcccggaagaagaagtcgccatctcaccgaaagaccgtggttactactttggcgatggtctgtatgaagtgttccgtatctaccagggccgcctgtttgaaaaagaaagccacctggcacgtctgcagcgtaccgcaaaggacctgcgtattaccacgccggttacgctggaagaactgtctggtcagctggaacaactgaccgtcgaaaacggcaccaaaacgggtattctgtatattcagatcacccgtggcgcagcaccgcgtacgcatagtttcccgtccgaaggtaccaagccggtggttatggcgtactgccaggatatggaacgcccgaccgaacagctggaacaaggcgtggcagctattacgggtgatgacatccgttggctgcgctgtgacctgaaaaccctgaacctgctgccgaatgttctggctaagcaggcggccgcagatcaagaagcggacgaaattatctttcaccgttccggcatcgtgaccgaatgcagctctaacaatgttatgatggtcaaagatggcattgttcgcacccatccggctaaccacctgattctgcatggtatcacgcgtgccgtcgtgctgcgtctgctgcatcagcaagatatcccggtcgaagaagccccgttcaccctgaaagaactgggctcagcagacgaagtttttattacgggtaccacgtcggaagtgaccccggttacggaaattgatggcatcccggtcggcaaaggtattccgggtccggtgacccgtaagatccagcaagcgttcgaagctgcgattaatctcgag

SEQ ID NO: 68 - protein, 284 aa, Paenibacillus larvae
MEASPILYLYNNHIVPEEEVAISPKDRGYYFGDGLYEVFRIYQGRLFEKESHLARLQRTAKDLRITTPVTLEELSGQLEQLTVENGTKTGILYIQITRGAAPRTHSFPSEGTKPVVMAYCQDMERPTEQLEQGVAAITGDDIRWLRCDLKTLNLLPNVLAKQAAADQEADEIIFHRSGIVTECSSNNVMMVKDGIVRTHPANHLILHGITRAVVLRLLHQQDIPVEEAPFTLKELGSADEVFITGTTSEVTPVTEIDGIPVGKGIPGPVTRKIQQAFEAAINLE

SEQ ID NO: 69 - DNA, 858 bp, Artificial Sequence
  Polynucleotide having modified codons, which encodes D-aminotransferase derived from Ruminococcaceae bacterium
catatgtggaagaatgttggctactataatggcacgatgggtccgctggaagaaatgacggtcccgatgggtgatcgcgctctgtactttggtgatggtatttatgaagcgacctgcgtcgccaaccgtgtgccgtttgcactggatgaccatctggatcgtatgtacaattctctgcgcctgctggaaattccgttcacgatggaacgcgaccaggttaaagctgaactgcaaaaggtcatcgatgcggccgaagactccccgattcattttctgtattggcagatctcacgtggcgtggcaatgcgcaaccacccgttcccggctaataccgaaccgacgctgctgatttatgttaaaccgcataccatgaagtctatggataaaccgtacaagctgattagtatggaagacatccgttttaaactgtgcaacattaagacgctgaatctgatcccgtcggtgctggcaaaccagcgcgctgttgaacatggctgcgatgaagcagtcctgcaccgtggtagccgcgtgaccgaatgtgcccactccaatatttcaatcctgaaagatggcgtgctgcaaaccgcgccgacggacgaactgattctgccgggtatcacgcgtaaacatctgctggcactggctaaggaacacggcatttcggttctggaaaaaccgtttagcatggtcgaactgatgaacgccgatgaagtgatcgttaccagctctagtgcgctgtgtatgaaagccgaatcaattgatggtatcccggttggcggtaaagacccgcagcgtctgaagctgctgcaagatgcgtacctggaaaaattccagcgcgaaacccaaccgaaactcgag

SEQ ID NO: 70 - protein, 285 aa, Ruminococcaceae bacterium
MWKNVGYYNGTMGPLEEMTVPMGDRALYFGDGIYEATCVANRVPFALDDHLDRMYNSLRLLEIPFTMERDQVKAELQKVIDAAEDSPIHFLYWQISRGVAMRNHPFPANTEPTLLIYVKPHTMKSMDKPYKLISMEDIRFKLCNIKTLNLIPSVLANQRAVEHGCDEAVLHRGSRVTECAHSNISILKDGVLQTAPTDELILPGITRKHLLALAKEHGISVLEKPFSMVELMNADEVIVTSSSALCMKAESIDGIPVGGKDPQRLKLLQDAYLEKFQRETQPKLE

SEQ ID NO: 71 - DNA, 864 bp, Artificial Sequence
  Polynucleotide having modified codons, which encodes D-aminotransferase derived from Robiginitalea biformata
catatgccgcacccgattgactacccgaagaaagtttacctgaatggcgaaatcctggacgcagaagaagcccgcatctccgtgtttgaccgtggctttctgttcggcgatggtatttatgaagttatggcccgtatcggcggtcgtttctttcgccaggcggatcacatggcccgcatgcaaagctgcctggaaaaaattgctatcccgtttgacgcgtctcgtctggaagcagaaattccggctctgctggaagcaagcggtctggccggtcaggattgtctgctgtatctgcaagtcacgcgtggcaccgcaccgcgtcagcatgcattcccgccggacgcacaaccgaccgctatgatgtacgcgtggccgaaaacgctgccggaagtcgaaacctcacgtgcgtcggtgatcacgcgcgaagattttcgttggcaccgctgcgacattaagagcacctctctgctgggtaacatcctgagtaatcaggaagcggccagtaactcctgctatgaaacgattttcatccgtgatggccgcgttaccgaagcatcacattgtaacgtgtttttcgtccgtggtgaagtggtttacacgcacccggcggataccaatattctggacggcattatccgtcgcgtcgtgctggaactgtgccgtgaactgggtctggaagtgcgtctggaaggtgttccggccggtcaggtccgtcaaatggatgaagcctttctgaccggcacgtccacccaggtgatggcaattgctcgcgttgacggcgaagcttgttaccaggaagcaccgggtccggtgacccgtcgcatccaagaagccttccgcgaagcaaaaaagggtgaactcgag

SEQ ID NO: 72 - protein, 287 aa, Robiginitalea biformata
MPHPIDYPKKVYLNGEILDAEEARISVFDRGFLFGDGIYEVMARIGGRFFRQADHMARMQSCLEKIAIPFDASRLEAEIPALLEASGLAGQDCLLYLQVTRGTAPRQHAFPPDAQPTAMMYAWPKTLPEVETSRASVITREDFRWHRCDIKSTSLLGNILSNQEAASNSCYETIFIRDGRVTEASHCNVFFVRGEVVYTHPADTNILDGIIRRVVLELCRELGLEVRLEGVPAGQVRQMDEAFLTGTSTQVMAIARVDGEACYQEAPGPVTRRIQEAFREAKKGELE

SEQ ID NO: 73 - DNA, 855 bp, Artificial Sequence
  Polynucleotide having modified codons, which encodes D-aminotransferase derived from Thiobacillus denitrificans
catatgagtgtttacctgaatggtcgctttcagccgctggccgaagcaacgattccggttctggaccgtggttttgtgtttggtgatggtgtttatgaactggttccggtctactcccgtaaaccgtttcgcctggatgcgcatctgacccgtctgcagcactcactggacggcattcgcctgtcgaacccgcataccccgacgcaatggcgtgatctgattcagcacctgatcgccgaacaggactttgatgaccaaagcgtctatatccaggtgacccgtggtacggcaccgcgcgatcatgcattcccggtgggtgttccgccgaccgtgtttatgttcgcacaagcactggttaccgcaacgccggcacagaaagctgtcggtgtgtgcgcagtgagcgcagttgataaccgttggctgcgctgtgacattaaggcaatctctctgctgccgaatattctgctgcgtcagcaagcaaccgatgctggttgcgcggaaacggtgatgtttcgtgacggtttcctgaccgaaggtgcagcaagtaacatcttcgtggttaaagatggtgtcctgctgacgccgccgagctctaatctgatgctgaccggcgttacgcacgatgtcgtgctggaactggcagctaccctgggtgttccggtcgaaattcgcgcaatcgctgaagcggaagtgcgtcgcgccgatgaactgtggatgaccagttccacgaaggaaattatgccgatcgttatgctggatggtgcaccggtcggcggtggcgctccgggtccgctggcgcagaaatttgacgcggcctacgcagctttcaagcgtgaagttatgcgcgcgctcgag

SEQ ID NO: 74 - protein, 284 aa, Thiobacillus denitrificans
MSVYLNGRFQPLAEATIPVLDRGFVFGDGVYELVPVYSRKPFRLDAHLTRLQHSLDGIRLSNPHTPTQWRDLIQHLIAEQDFDDQSVYIQVTRGTAPRDHAFPVGVPPTVFMFAQALVTATPAQKAVGVCAVSAVDNRWLRCDIKAISLLPNILLRQQATDAGCAETVMFRDGFLTEGAASNIFVVKDGVLLTPPSSNLMLTGVTHDVVLELAATLGVPVEIRAIAEAEVRRADELWMTSSTKEIMPIVMLDGAPVGGGAPGPLAQKFDAAYAAFKREVMRALE

SEQ ID NO: 75 - DNA, 867 bp, Artificial Sequence
  Polynucleotide having modified codons, which encodes D-aminotransferase derived from Rhodobacter sphaeroides
catatgagccgtaccgtttatgtgaatggcgaatacctgccggaagaagaagcgaccgttagcatttttgaccgtggtttcctgatggcggatggcgtgtatgaagttacctccgtcctgggcggtaaactgattgattttccgggtcatgcggcccgtctggaacgcagtctgaacgaactggaaatggctgttccgatgtccaccgaagaactgctggaagttcaccgtgaactggtcgcgcgcaacggcattgaagaaggtctggtgtacctgcagatcacccgtggcaatccgggtgatcgcgactttgctttcccgccggcggataccaaaccgacggtggttctgttcacgcagagcaagccgggtctggcagctaatccggtggcacaagttggtatcaaagttatttctatcccggatattcgttggggccgtcgcgacatcaaaaccgtccaactgctgtatccgagcatggcaaagatggcggccaaaaaggcccatgttgatgacgcatggtttgtggaagatggcttcgttaccgaaggcacgtcaaacaatgtctacattgtgaaaggcggtaaaatcgtgacccgtgatctgtcgaacgacattctgcacggtatcacgcgcgcagctgtcgtgcgttttgcacgcgaagctcagatggaagttgaagaacgcccgttcaccattgaagaagcgcaaggtgccgatgaagcatttttcacgagcgcgtctgcctttgtgctgccggttgtcgaaatcgacggcaaagccgtcggcaccggtacgccgggtccggtggcagcacgtctgcgtgaactgtatctggaagaaagtctgaaggcagctgtgctcgag

SEQ ID NO: 76 - protein, 288 aa, Rhodobacter sphaeroides
MSRTVYVNGEYLPEEEATVSIFDRGFLMADGVYEVTSVLGGKLIDFPGHAARLERSLNELEMAVPMSTEELLEVHRELVARNGIEEGLVYLQITRGNPGDRDFAFPPADTKPTVVLFTQSKPGLAANPVAQVGIKVISIPDIRWGRRDIKTVQLLYPSMAKMAAKKAHVDDAWFVEDGFVTEGTSNNVYIVKGGKIVTRDLSNDILHGITRAAVVRFAREAQMEVEERPFTIEEAQGADEAFFTSASAFVLPVVEIDGKAVGTGTPGPVAARLRELYLEESLKAAVLE

SEQ ID NO: 77 - DNA, 867 bp, Artificial Sequence
  Polynucleotide having modified codons, which encodes D-aminotransferase derived from Oceanibulbus indolifex
catatgcgcaccgtttatgtcaatggcgaatacctgccggaagaccaagcaaagatttcaattttcgaccgtggttttctgatggctgatggcgtctatgaagtgaccagcgttctggacggcaaactgattgcctttgatggtcatgcagaacgtctgacgcgctctatgaacgaactggatatgcgtgcaccggctaccacggaagaactgctggaaatccaccgtgaactggttcgcctgaatgacattaaagatggcctgatctacctgcaggtcacccgtggcagcgatggtgaccgcgattttgctttcccggatccggaaaccacggcgccgtcactggttctgttcacccaatcgaagccgggtctggcagacaacccggcagcccgtgatggtattcgcgttattagtatcgaagacattcgctggggtcgtcgcgatatcaaaaccgtgcagctgctgtatccgtccatgggcaagatgatggcaaaaaaggctggtgtggaagacgcttggatggtcgaagatggctttgtgaccgagggtacgagcaacaatgcatacattgttaaagacggcaagatcgtcacccgtgaaacgagctctgatattctgcatggtatcacccgtaaagcagtcctggaactggcgcgcgaagcacagatgcaagtggaagaacgcaatttcacgattgaagaagcacagcaagctgacgaagcgtttgtgaccagtgcgtccgccttcgttacgccggtggttgaaatcgatggcgccaaagtgggctctggtaccccgggcaagctggcaccgcgtctgcgtgaaatttatctggaagaaatgcgtaaaacggcgatcctcgag

SEQ ID NO: 78 - protein, 288 aa, Oceanibulbus indolifex
MRTVYVNGEYLPEDQAKISIFDRGFLMADGVYEVTSVLDGKLIAFDGHAERLTRSMNELDMRAPATTEELLEIHRELVRLNDIKDGLIYLQVTRGSDGDRDFAFPDPETTAPSLVLFTQSKPGLADNPAARDGIRVISIEDIRWGRRDIKTVQLLYPSMGKMMAKKAGVEDAWMVEDGFVTEGTSNNAYIVKDGKIVTRETSSDILHGITRKAVLELAREAQMQVEERNFTIEEAQQADEAFVTSASAFVTPVVEIDGAKVGSGTPGKLAPRLREIYLEEMRKTAILE

SEQ ID NO: 79 - DNA, 852 bp, Artificial Sequence
  Polynucleotide having modified codons, which encodes D-aminotransferase derived from Lactobacillus salivarius
catatgaaacaagtcggctactataacggcaccatcgctgacctgaatgaactgaaagtcccggcaacggatcgcgccctgtatttcggtgatggttgttatgatgcgaccacgtttaaaaacaatgtggcgttcgccctggaagatcatctggaccgtttttacaactcttgccgcctgctggaaattgatttcccgctgaatcgtgacgaactgaaagaaaagctgtatgcggttattgatgccaacgaagtcgacaccggtatcctgtactggcagacgtcccgtggctcaggtctgcgcaaccatatttttccggaagattcccaaccgaatctgctgatctttaccgccccgtatggcctggttccgttcgatacggaatacaaactgatctcacgtgaagacacccgctttctgcactgtaacattaagacgctgaacctgctgccgaatgtcatcgcatcgcagaaagctaatgaaagccattgccaagaagtggttttccaccgtggcgatcgcgtcaccgaatgtgcacattctaacattctgatcctgaaagatggtgtgctgtgcagtccgccgcgtgacaatctgattctgccgggcatcaccctgaagcacctgctgcagctggcaaaagaaaacaatattccgacctcggaagctccgttcacgatggatgacctgcgtaacgcggatgaagtcattgtgagctctagtgcatgcctgggtatccgtgcagtggaactggatggtcagccggttggcggtaaagacggcaagaccctgaaaatcctgcaagacgcgtatgcgaaaaaatacaatgccgaaaccgttagtcgcctcgag

SEQ ID NO: 80 - protein, 283 aa, Lactobacillus salivarius
MKQVGYYNGTIADLNELKVPATDRALYFGDGCYDATTFKNNVAFALEDHLDRFYNSCRLLEIDFPLNRDELKEKLYAVIDANEVDTGILYWQTSRGSGLRNHIFPEDSQPNLLIFTAPYGLVPFDTEYKLISREDTRFLHCNIKTLNLLPNVIASQKANESHCQEVVFHRGDRVTECAHSNILILKDGVLCSPPRDNLILPGITLKHLLQLAKENNIPTSEAPFTMDDLRNADEVIVSSSACLGIRAVELDGQPVGGKDGKTLKILQDAYAKKYNAETVSRLE

SEQ ID NO: 81 - DNA, 861 bp, Artificial Sequence
  Polynucleotide having modified codons, which encodes D-aminotransferase derived from ID910 disclosed in WO2009/088482A1
catatggcttactcgctgtggaatgaccagattgttgaagaaggctctatcgctgtctccccggaagatcgtggctaccagttcggtgacggtatttatgaagtcatcaaggtgtacaacggcaatatgtttaccgcgcaggaacatattgatcgtttctatgcaagcgctgaaaaaattcgcctggttatcccgtacacgaaagatgtcctgcataagctgctgcacgaactgattgaaaagaacaatctggacaccggccatgtctattttcaaatcacgcgtggtgccaacagccgcaatcacgtgttcccggatgcgtctgttccggccgtcctgaccggcaacgttaaagcaggtgaacgtgcttacgaaaattttgaaaaaggcgtcaaggcaacgttcgtggaagatattcgttggctgcgctgcgacatcaaatccctgaacctgctgggtgcggtgctggccaaacaggaagcggccgaaaagggctgctatgaagcgattctgcatcgtggtgatatcgtgaccgaatgtagctctgccaacgtttatggcattaaagacggcaagctgtacacccacccggcgaacaattttattctgaatggtatcacgcgccaagtgattctgaagtgtgcagaagaaatcagtctgccggtggttgaagaaccgatgaccaaagctgatctgctgacgatggacgaaattatcgtgagttccgtttcatcggaagtcaccccggtgattgatgttgacggcaaccaaatcggtgcaggtgttccgggtgaatggacccgcaaactgcagcaagcattcgaagctaagctgccgctgtcactgaatacgaaactcgag

SEQ ID NO: 82 - protein, 286 aa, Unknown
  Obtained from environmental sample (ID910 disclosed in WO2009/088482A1)
MAYSLWNDQIVEEGSIAVSPEDRGYQFGDGIYEVIKVYNGNMFTAQEHIDRFYASAEKIRLVIPYTKDVLHKLLHELIEKNNLDTGHVYFQITRGANSRNHVFPDASVPAVLTGNVKAGERAYENFEKGVKATFVEDIRWLRCDIKSLNLLGAVLAKQEAAEKGCYEAILHRGDIVTECSSANVYGIKDGKLYTHPANNFILNGITRQVILKCAEEISLPVVEEPMTKADLLTMDEIIVSSVSSEVTPVIDVDGNQIGAGVPGEWTRKLQQAFEAKLPLSLNTKLE

SEQ ID NO: 83 - DNA, 870 bp, Artificial Sequence
  Polynucleotide having modified codons, which encodes D-aminotransferase derived from ID906 disclosed in WO2009/088482A1
catatgcgcaccgtttacgtgaatggcaagtttctgccggaagcggaaggcatggtttcgatttttgaccgtggttttacgatgagcgacagcgtgtatgaagttaccggcgtggttgcaggtaaaatgctggattttgacccgcacatggctcgtctgcagcgcagcatgtctgaactgggtatgccgaaaggcccgggtgcagatgctctgaaagcaattcacctgaagatgatcgaactgaacaagctggatgaaggcctggtgtacctgcagattacccgtggtgttcaagatcgcaactttgaactgccggcggccgacaatccgctgaccatcgtgctgttcacgcaggcacgtccggtcgtggaatccgcacaagccgaacgcggtattaaagttatcagtctgccggatctgcgttggcatcgctccgacattaaaaccacgcagctgctgtatgcgtgcctggccaaggatgcagctaaaaagcgtggctgtgatgacgcatggctggtccgtgacggtctggtgaccgaaggtagcgcaaacaatgctttcattgttacgcgtgatggcctggtcatcacccgcgacctgtctacggaactgctgccgggtattacccgtaaacgcatcgtggaactggcggccaatcacggctatcgtctggaacaacgcgcgtttacgattgcagaagctaaggcagctgtcgaagcgtttatcaccagcgccacgcagttcgttatgccggttgtcgaaattgatggcgcggccatcggccaaggtaaaccgggcaagttctcagtggcgctgcgtaaactgtacattgaagaaaccctgtcgcgcgccggcatccgtcgcctcgag

SEQ ID NO: 84 - protein, 289 aa, Unknown
  Obtained from environmental sample (ID906 disclosed in WO2009/088482A1)
MRTVYVNGKFLPEAEGMVSIFDRGFTMSDSVYEVTGVVAGKMLDFDPHMARLQRSMSELGMPKGPGADALKAIHLKMIELNKLDEGLVYLQITRGVQDRNFELPAADNPLTIVLFTQARPVVESAQAERGIKVISLPDLRWHRSDIKTTQLLYACLAKDAAKKRGCDDAWLVRDGLVTEGSANNAFIVTRDGLVITRDLSTELLPGITRKRIVELAANHGYRLEQRAFTIAEAKAAVEAFITSATQFVMPVVEIDGAAIGQGKPGKFSVALRKLYIEETLSRAGIRRLE

SEQ ID NO: 85 - DNA, 885 bp, Artificial Sequence
  Polynucleotide having modified codons, which encodes D-aminotransferase derived from ID884 disclosed in WO2009/088482A1
catatggtttacctgaatggccgtttcgtgccgatggaagaagcagttgtcccgattgaagaccgtggttatcagtttgccgatggtgtttatgatgttctgaaatttcatggtcgttgcgcagtccgtctgaccgcacacctggaacgtctggctgaaagttgtgcgggcctgcgcattgaaggtgtgccgagtgcacagggttggcgttccattatcgctgaactggcggaacgctcagaactgggtcataccttcgatgacacggttattctgtatatccaagtgacccgtggtgttgcatcgcgcattcactactttccggaaccgcgtccgaaaccgacggtgctggcgtatttcaaggcagcaccggtttacccggaagctctgcgtcgcgatggtgcgagcgtgatcaccatgccggacgaacgttggggtcgctgccatattaaatctatcgctctgctgccggtggttctggccaagcaggcagctcgtgaagctggtgcactggaagcactgctggttcgtgatggtattgtcaccgaaggcggtgccagtaacgcattttgtgtccgtcaaggcgtgatcttcacccacccggaaggtccgcgtattctgtccggcgtgacgcgcggtatcgttctggatgcggcccgtcgcctgggcattgaagtccgtgaagaaccggtgccgatcgaagaatttcgcgcagctgacgaagcgttcctgagctctaccacgatgaacgtcatgccggtgacccgcattaatggtgcaccggtcggttcaggtgcagttggtgaagtcacgcgttcgattgcggccgcagttgaagaactgatccgcgaagaacatgaagccaatgcatctctgggtctcgag

SEQ ID NO: 86 - protein, 294 aa, Unknown
  Obtained from environmental sample (ID884 disclosed in WO2009/088482A1)
MVYLNGRFVPMEEAVVPIEDRGYQFADGVYDVLKFHGRCAVRLTAHLERLAESCAGLRIEGVPSAQGWRSIIAELAERSELGHTFDDTVILYIQVTRGVASRIHYFPEPRPKPTVLAYFKAAPVYPEALRRDGASVITMPDERWGRCHIKSIALLPVVLAKQAAREAGALEALLVRDGIVTEGGASNAFCVRQGVIFTHPEGPRILSGVTRGIVLDAARRLGIEVREEPVPIEEFRAADEAFLSSTTMNVMPVTRINGAPVGSGAVGEVTRSIAAAVEELIREEHEANASLGLE

(19) United States
(12) Patent Application Publication
(10) Pub. No.: US 2012/0295314 A1
(43) Pub. Date: Nov. 22, 2012

(54) METHOD FOR PRODUCING MONATIN

(75) Inventors: Masakazu Sugiyama, Kanagawa (JP); Yasuaki Takakura, Kanagawa (JP); Mika Moriya, Kanagawa (JP); Yusuke Hagiwara, Kanagawa (JP); Eri Tabuchi, Kanagawa (JP)

(73) Assignee: Ajinomoto Co., Inc., Tokyo (JP)

(21) Appl. No.: 13/455,381

(22) Filed: Apr. 25, 2012

Related U.S. Application Data
(60) Provisional application No. 61/478,679, filed on Apr. 25, 2011.

(51) Int. Cl. C12P 17/10; C07H 21/00; C12N 9/10

(52) U.S. Cl. 435/106; 435/193; 536/23.2

(57) ABSTRACT

The present invention provides a method of producing 2R,4R-Monatin with a good yield using inexpensive L-Trp rather than expensive D-Trp as a stating material. Specifically, the present invention provides a method for producing 2R,4R-Monatin or a salt thereof, comprising:
(1) contacting L-tryptophan with a deamination enzyme to form indole-3-pyruvate;
(2) contacting the indole-3-pyruvate and pyruvate with an aldolase to form 4R-IHOG; and
(3) contacting the 4R-IHOG with a D-aminotransferase in the presence of a D-amino acid to form the 2R,4R-Monatin; and the like. In (3), it is preferable to use a D-aminotransferase having no or low ability to form D-tryptophan from indole-3-pyruvate.

### CROSS-REFERENCE TO RELATED APPLICATIONS

This application claims the benefit of priority from U.S. provisional Patent Application No. 61/478,679, filed on Apr. 25, 2011, the entire contents of which are incorporated herein by reference.

### TECHNICAL FIELD

The present invention relates to a method for producing Monatin, and the like.

### BACKGROUND ART

Monatin [4-(indole-3-yl-methyl)-4-hydroxy-glutamic acid] is a compound that is one of amino acids contained in roots of Schlerochitom ilicifolius that is a shrub in South Africa and is particularly expected as a low calorie sweetener because of having sweetness one thousand and several hundreds times sweeter than sucrose (see Patent Document 1). The Monatin has asymmetric carbon atoms at positions 2 and 4, and a naturally occurring stereoisomer of Monatin is a 2S,4S-isomer. Naturally non-occurring three stereoisomers have been synthesized by organic chemistry processes. All of these stereoisomers are excellent in sweetness, and expected to be used as the sweeteners.

Several methods have been reported as the methods for producing the Monatin (see, e.g., Patent Document 2). However, all of the reported methods require multiple steps, and thus, it is required to improve a synthetic yield of the Monatin.

Specifically, for the method for producing the Monatin, the following method for producing 2R,4R-Monatin by synthesizing indole-3-pyruvate (hereinafter referred to as “IPA” as needed) from L-tryptophan (L-Trp), synthesizing 4R form of 4-(indol-3-yl-methyl)-4-hydroxy-2-oxoglutaric acid (hereinafter referred to as “4R-IHOG” as needed) from the resulting IPA and pyruvate, and subsequently subjecting the obtained 4R-IHOG to an oximation reaction, a reduction reaction and an epimerization-crystallization method is known (conventional method (1)) (see Patent Document 2).

However, an aldolase step (second step) is an equilibrium reaction, and thus, a satisfactory yield is not always obtained in this reaction.

### Conventional Method (1) for Producing 2R,4R-Monatin

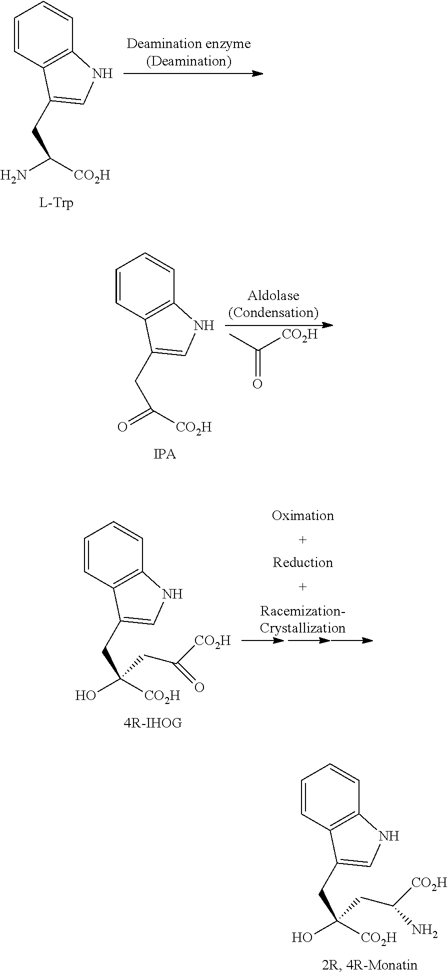

In order to improve the yield of the 2R,4R-Monatin, the method for producing the 2R,4R-Monatin by a one-pot enzymatic reaction was invented (see Patent Documents 3 to 6). For example, the method for producing the 2R,4R Monatin by the one-pot enzymatic reaction using D-tryptophan (D-Trp) as a starting material as well as a D-aminotransferase and an aldolase is known (conventional method (2)) (see Patent Documents 7 and 8).

However, expensive D-Trp is used as the starting material in this method, and thus, it has been required to inexpensively perform the one-pot enzymatic reaction.

### Conventional Method (2) for Producing 2R,4R-Monatin

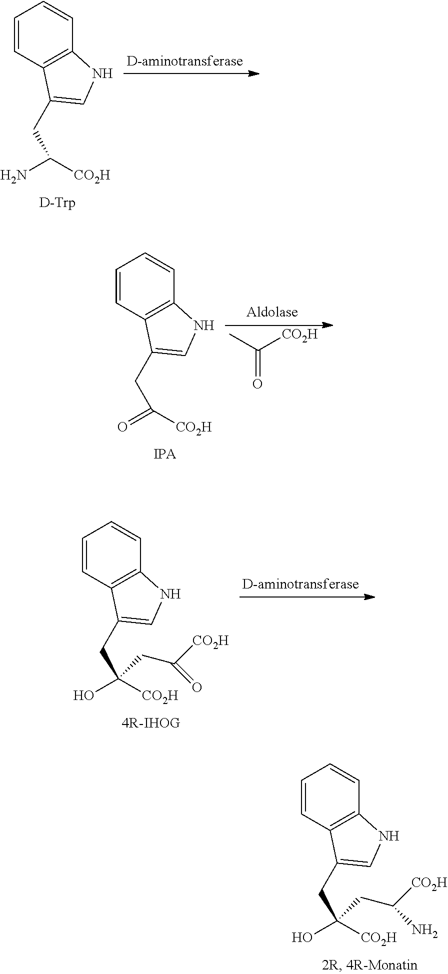

### PRIOR ART LITERATURE

### Patent Document

Patent Document 1: JP Sho-64-25757-A
Patent Document 2: International Publication WO2003/059865
Patent Document 3: International Publication WO2007/133184
Patent Document 4: International Publication WO2005/042756
Patent Document 5: US Patent Application Publication No. 2006/0252135 Specification
Patent Document 6: US Patent Application Publication No. 2008/020434 Specification
Patent Document 7: International Publication WO2003/091396
Patent Document 8: US Patent Application Publication No. 2005/0244937 Specification

### SUMMARY OF INVENTION

### Problem to be Solved by the Invention

The object of the present invention is to provide a method for inexpensively producing Monatin with a good yield.

### Means for Solving Problem

As a result of an extensive study, the present inventors have found that 2R,4R-Monatin can be produced with a good yield from inexpensive L-Trp utilizing a certain enzymatic reaction, and completed the present invention.

Accordingly, the present invention is as follows.

[1] A method for producing 2R,4R-Monatin or a salt thereof, comprising:
(1) contacting L-tryptophan with a deamination enzyme to form indole-3-pyruvate;
(2) contacting the indole-3-pyruvate and pyruvate with an aldolase to form 4R-IHOG; and
(3) contacting the 4R-IHOG with a D-aminotransferase in the presence of a D-amino acid to form the 2R,4R-Monatin.
[2] The method of [1], wherein the steps (1)-(3) are carried out in one reactor.
[3] The method of [1], wherein the deamination enzyme is a deaminase that is capable of acting on the L-tryptophan to form the indole-3-pyruvate.
[4] The method of [1], wherein the D-aminotransferase has no or low ability to form D-tryptophan from the indole-3-pyruvate.
[5] The method of [4], wherein the D-aminotransferase is derived from a microorganism belonging to genus Achromobacter, genus Agrobacterium, genus Bacillus, genus Coprococcus, genus Geobacillus, genus Halothiobacillus, genus Lactobacillus, genus Oceanibulbus, genus Paenibacillus, genus Rhodobacter, genus Robiginitalea, or genus Thiobacillus.
[6] The method of [5], wherein the D-aminotransferase is derived from a microorganism belonging to Achromobacter xylosoxidans, Agrobacterium radiobacter, Bacillus halodurans, Bacillus megaterium, Bacillus macerans, Bacillus proteiformans, Coprococcus comes, Geobacillus sp., Geobacillus toebii, Halothiobacillus neapolitanus, Lactobacillus salivarius, Oceanibulbus indolifex, Paenibacillus larvae, Rhodobacter sphaeroides, Robiginitalea biformata, or Thiobacillus denitrificans.
[7] The method of [4], wherein the D-aminotransferase comprises a mutation of one or more amino acid residues selected from the group consisting of the amino acid residues at positions 87, 100, 117, 145, 157, 240, 243 and 244 in the amino acid sequence represented by SEQ ID NO:2.
[8] The method of [7], wherein the mutation of the amino acid residue is selected from the group consisting of:
i) the substitution of histidine at position 87 with arginine;
ii) the substitution of asparagine at position 100 with threonine;
iii) the substitution of lysine at position 117 with arginine or glutamine;
iv) the substitution of isoleucine at position 145 with valine;
v) the substitution of lysine at position 157 with arginine, glutamine or threonine;
vi) the substitution of serine at position 240 with threonine;
vii) the substitution of serine at position 243 with asparagine; and
viii) the substitution of serine at position 244 with lysine.
[9] The method of [1], further comprising contacting a keto acid with a decarboxylase to degrade the keto acid, wherein the keto acid is formed from the D-amino acid due to action of the D-aminotransferase.
[10] The method of [9], wherein the D-amino acid is D-aspartate.
[11] The method of [10], further comprising contacting oxaloacetate with an oxaloacetate decarboxylase to irreversibly form pyruvate, wherein the oxaloacetate is formed from the D-aspartate by action of the D-aminotransferase.
[12] The method of [11], wherein at least part of the pyruvate used in the formation of the 4R-IHOG is from pyruvate formed from the oxaloacetate due to action of the oxaloacetate decarboxylase.
[13] The method of [1], wherein the salt is a sodium salt, a potassium salt, a magnesium salt or a calcium salt.
[14] A D-aminotransferase that has an ability to form 2R,4R-Monatin from 4R-IHOG in the presence of a D-amino acid, and that has no or low ability to form D-tryptophan from indole-3-pyruvate.
[15] The D-aminotransferase of [14], comprising a mutation of one or more amino acid residues selected from the group consisting of the amino acid residues at positions 87, 100, 117, 145, 157, 240, 243 and 244 in the amino acid sequence represented by SEQ ID NO:2.
[16] The D-aminotransferase of [17], wherein the mutation of the amino acid residue is selected from the group consisting of:
i) the substitution of histidine at position 87 with arginine;
ii) the substitution of asparagine at position 100 with threonine;
iii) the substitution of lysine at position 117 with arginine or glutamine;
iv) the substitution of isoleucine at position 145 with valine;
v) the substitution of lysine at position 157 with arginine, glutamine or threonine;
vi) the substitution of serine at position 240 with threonine;
vii) the substitution of serine at position 243 with asparagine; and
viii) the substitution of serine at position 244 with lysine.
[17] A polynucleotide encoding the D-aminotransferase of [14].
[18] A method for producing 2R,4R-Monatin or a salt thereof, comprising the following two steps carried out in one reactor:
(1′) contacting indole-3-pyruvate and pyruvate with an aldolase to form 4R-IHOG; and
(2′) contacting the 4R-IHOG with a D-aminotransferase in the presence of a D-amino acid to form the 2R,4R-Monatin.

### Effect of the Invention

The method of the present invention can produce 2R,4R-Monatin with a good yield from L-Trp that is an inexpensive material. The method of the present invention can also produce 2R,4R-Monatin with a good yield from L-Trp by performing a deamination reaction by a deamination enzyme, a condensation reaction by an aldolase and an amination reaction by a D-aminotransferase in one reactor (one-pot enzymatic reaction). The method of the present invention can further produce 2R,4R-Monatin with a very good yield from L-Trp by using a D-aminotransferase that is inert for IPA (IPA-inert).

### BRIEF DESCRIPTION OF DRAWINGS

FIG. 1 is a view showing an outline of the production method of the present invention. L-Trp: L-tryptophan; IPA: indole-3-pyruvate; PA: pyruvate; 4R-IHOG: 4R-4-(indole-3-yl-methyl)-4-hydroxy-2-oxoglutaric acid; 2R,4R-Monatin: 2R,4R-4-(indole-3-yl-methyl)-4-hydroxy-glutamic acid.

FIG. 2 is a view showing one example of the production method of the present invention. Abbreviations are the same as in FIG. 1. The D-aminotransferase is preferably one having an ability to form 2R,4R-Monatin from 4R-IHOG in the presence of a D-amino acid, and having no or low ability to form D-Trp from IPA.

FIG. 3 is a view showing one preferable example of the production method of the present invention. The abbreviations are the same as above.

FIG. 4 is a view showing one example of the production method of the present invention. D-alanine: D-Ala. The other abbreviations are the same as above. The D-aminotransferase is preferably one having an ability to form 2R,4R-Monatin from 4R-IHOG in the presence of a D-amino acid and having no or low ability to form D-Ala from PA.

FIG. 5 is a view showing one preferable example of the production method of the present invention. The abbreviations are the same as above.

FIG. 6 is a view showing one example of the production method of the present invention. The abbreviations are the same as above.

FIG. 7 is a view showing one preferable example of the production method of the present invention. The abbreviations are the same as above.

FIG. 8 is a view showing one example of the production method of the present invention. The abbreviations are the same as above.

FIG. 9 is a view showing one example of the production method of the present invention. D-Asp: D-aspartic acid; OAA: oxaloacetic acid. The other abbreviations are the same as above.

FIG. 10 is a view showing one example of the production method of the present invention. The abbreviations are the same as above.

FIG. 11 is a view showing one example of the production method of the present invention. The abbreviations are the same as above.

FIG. 12 is a view showing one preferable example of the production method of the present invention. The abbreviations are the same as above.

FIG. 13 is a view showing transition of D-Trp and 2R,4R-Monatin. D-Trp: D-tryptophan; RR-Monatin: 2R,4R-Monatin [2R,4R-4-(indole-3-yl-methyl)-4-hydroxy-glutamic acid].

FIG. 14 is a view showing transition of indole compounds over time. The abbreviations are the same as above. The abbreviations are the same as above. RR-Monatin: 2R,4R-4-(indole-3-yl-methyl)-4-hydroxy-glutamic acid; RS-Monatin: 2R,4S-4-(indole-3-yl-methyl)-4-hydroxy-glutamic acid.

FIG. 15 is a view showing transition of indole compounds over time. The abbreviations are the same as above.

### BEST MODES FOR CARRYING OUT THE INVENTION

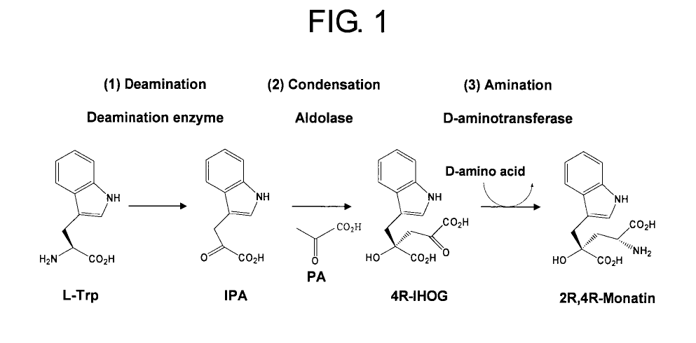

The present invention provides a method for producing 2R,4R-Monatin or a salt thereof. The method of the present invention comprises the following (1) to (3) (see FIG. 1).

(1) Contacting L-tryptophan (L-Trp) with a deamination enzyme to form indole-3-pyruvate (IPA) (deamination reaction)
(2) Contacting the indole-3-pyruvate (IPA) and pyruvate (PA) with an aldolase to form 4R-IHOG (condensation reaction) and
(3) Contacting the 4R-IHOG with a D-aminotransferase in the presence of a D-amino acid to form the 2R,4R-Monatin (amination reaction).

The above reactions (1) to (3) are performed by, for example, using enzymes or enzyme-producing microorganisms, or combinations thereof.

The aforementioned deamination reaction, condensation reaction and amination reaction may be progressed separately or in parallel. These reactions may be carried out in one reactor (e.g., one-pot enzymatic reaction). When these reactions are carried out in one reactor, these reactions can be carried out by adding substrates and enzymes sequentially or simultaneously. Specifically, when the aforementioned deamination reaction, condensation reaction and amination reaction are carried out, (1) L-Trp and the deamination enzyme or a deamination enzyme-producing microorganism, (2) the pyruvate and the aldolase or an aldolase-producing microorganism, and (3) the D-amino acid and the D-aminotransferase or a D-aminotransferase-producing microorganism may be added in one reactor sequentially or simultaneously. The enzyme-producing microorganism may produce two or more enzymes selected from the group consisting of the deamination enzyme, the aldolase and the D-aminotransferase.

### (1) Deamination Reaction

As used herein, the term “deamination enzyme” refers to an enzyme capable of forming IPA from L-Trp. The formation of IPA from L-Trp is essentially conversion of the amino group (—NH₂) in L-Trp to an oxo group (═O). Therefore, the enzymes that catalyze this reaction are sometimes termed as other names such as a deaminase, an oxidase, a dehydrogenase, or an L-aminotransferase. Therefore, the term “deamination enzyme” means any enzyme that can form IPA from L-Trp, and the enzymes having the other name (e.g., deaminase, oxidase, dehydrogenase or L-aminotransferase) which catalyze the reaction to form IPA from L-Trp are also included in the “deamination enzyme.”

Examples of the method for forming IPA from L-Trp using the deaminase or deaminase-producing microorganism capable of acting upon L-Trp to form IPA include the method disclosed in International Publication WO2009/028338. A general formula for the reaction catalyzed by the deaminase includes the following formula: Amino acid+H₂O→2-oxo acid+NH₃.

Examples of the method for forming IPA from L-Trp using the oxidase or oxidase-producing microorganism capable of acting upon L-Trp to form IPA include the methods disclosed in U.S. Pat. No. 5,002,963, John A. Duerre et al. (Journal of Bacteriology 1975, vol. 121, No. 2, p656-663), JP-Sho-57-146573-A, International Publication WO2003/056026 and International Publication WO2009/028338. The general formula for the reaction catalyzed by the oxidase includes the following formula: Amino acid+O₂+H₂O→2-Oxo acid+H₂O₂+NH₃. For the purpose of suppressing the degradation of the compound by hydrogen peroxide as the by-product produced at that time, a hydrogen peroxide-degrading enzyme such as a catalase may be added in the reaction solution.

An L-amino acid dehydrogenase can also be used as the method for forming IPA from L-Trp using the dehydrogenase or dehydrogenase-producing microorganism capable of acting upon L-Trp to form IPA. Examples of the reaction method using the L-amino acid dehydrogenase include the methods using the enzyme disclosed in Toshihisa Ohshima and Kenji Soda, Stereoselective biocatalysis: amino acid dehydrogenases and their applications. Stereoselective Biocatalysis (2000), 877-902. The general formula for the reaction catalyzed by the dehydrogenase includes the following formula: L-amino acid+NAD(P)+H₂O→2-Oxo acid+NAD(P)H+NH₃.

Examples of the method for forming IPA from L-Trp using the L-aminotransferase or L-aminotransferase-producing microorganism capable of acting upon L-Trp to form IPA include the methods disclosed in East Germany Patent DD 297190, JP Sho-59-95894-A, International Publication WO2003/091396 and US Patent Application Publication No. 2005/0282260 Specification. The general formula for the reaction catalyzed by the L-aminotransferase includes the following formula:

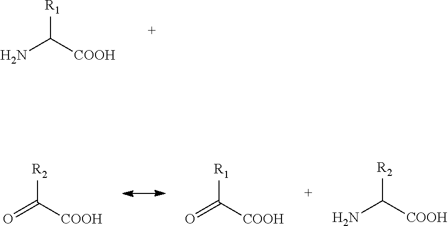

In addition, for the deamination enzyme used in the deamination reaction, the enzymes disclosed in the specifications of WO 2003/091396 and US Patent Application Publication No. 2005/0244937 may be used. For example, the following enzymes are used. As mentioned above, the following enzyme is abbreviated as the deamination enzyme such as deaminase, oxidase, dehydrogenase or L-aminotransferase, as long as it can form IPA from L-Trp.

EC 2.6.1.27: tryptophan aminotransferase (also termed L-phenylalanine-2-oxoglutarate aminotransferase, tryptophan transaminase, 5-hydroxytryptophan-ketoglutaric transaminase, hydroxytryptophan aminotransferase, L-tryptophan aminotransferase, L-tryptophan transaminase, and L-tryptophan: 2-oxoglutarate aminotransferase) which converts L-tryptophan and 2-oxoglutarate to indole-3-pyruvate and L-glutamate;

EC 1.4.1.19: tryptophan dehydrogenase (also termed NAD (P)-L-tryptophan dehydrogenase, L-tryptophan dehydrogenase, L-Trp-dehydrogenase, TDH and L-tryptophan: NAD(P) oxidoreductase (deaminating)) which converts L-tryptophan and NAD(P) to indole-3-pyruvate and NH3 and NAD(P)H;

EC 2.6.1.28: tryptophan-phenylpyruvate transaminase (also termed L-tryptophan-α-ketoisocaproate aminotransferase and L-tryptophan: phenylpyruvate aminotransferase) which converts L-tryptophan and phenylpyruvate to indole-3-pyruvate and L-phenylalanine;

EC 1.4.3.2: L-amino acid oxidase (also termed ophio-amino-acid oxidase and L-amino-acid: oxygen oxidoreductase (deaminating)) which converts an L-amino acid and H₂O and O₂ to a 2-oxo acid and NH₃ and H₂O₂; and

tryptophan oxidase which converts L-tryptophan and H₂O and O₂ to indole-3 pyruvate and NH₃ and H₂O₂.

For example, the L-amino acid oxidases are known which are derived from Vipera lebetine (sp P81375), Ophiophagus hannah (sp P81383), Agkistrodon rhodostoma (sp P81382), Crotalus atrox (sp P56742), Burkholderia cepacia, Arabidopsis thaliana, Caulobacter cresentus, Chlamydomonas reinitardtii, Mus musculus, Pseudomonas syringae, and Rhodococcus str. The tryptophan oxidases are known which are derived from, e.g., Coprinus sp. SF-1, Chinese cabbage with club root disease, Arabidopsis thaliana, and mammalian.

In addition, the tryptophan dehydrogenases are known which are derived from, e.g., spinach, Pisum sativum, Prosopis juliflora, pea, mesquite, wheat, maize, tomato, tobacco, Chromobacterium violaceum, and Lactobacilli.

In one embodiment, the contact of L-Trp with the deamination enzyme can be accomplished by allowing L-Trp and the deamination enzyme extracted from the deamination enzyme-producing microorganism (extracted enzyme) to coexist in the reaction solution. Examples of the deamination enzyme-producing microorganism include microorganisms that naturally produce the deamination enzyme and transformants that express the deamination enzyme. Specifically, examples of the extracted enzyme include a purified enzyme, a crude enzyme, an enzyme-containing fraction prepared from the above enzyme-producing microorganism, and a disrupted product of and a lysate of the above enzyme-producing microorganism.

In another embodiment, the contact of L-Trp with the deamination enzyme can be accomplished by allowing L-Trp and the deamination enzyme-producing microorganism to coexist in the reaction solution (e.g., culture medium).

The reaction solution used for the deamination reaction is not particularly limited as long as the objective reaction progresses, and for example, water and buffer are used. Examples of the buffer include Tris buffer, phosphate buffer, carbonate buffer, borate buffer and acetate buffer. When the deamination enzyme-producing microorganism is used in the production method of the present invention, the culture medium may be used as the reaction solution. Such a culture medium can be prepared using a medium described later.

A pH value for the deamination reaction is not particularly limited as long as the objective reaction progresses, and is, for example, pH 5 to 10, is preferably pH 6 to 9 and is more preferably pH 7 to 8.

A reaction temperature in the deamination reaction is not particularly limited as long as the objective reaction progresses, and is, for example, 10 to 50° C., is preferably 20 to 40° C. and is more preferably 25 to 35° C.

A reaction time period in the deamination reaction is not particularly limited as long as the time period is sufficient to form IPA from L-Trp, and is, for example, 2 to 100 hours, is preferably 4 to 50 hours and is more preferably 8 to 25 hours.

### (2) Condensation Reaction

As used herein, the term “aldolase” refers to an enzyme capable of forming 4R-IHOG from IPA and PA by an aldol condensation. The method for condensing IPA and PA by the aldolase to form 4R-IHOG is disclosed in, for example, International Publication WO2003/056026, JP 2006-204285-A, US Patent Application Publication No. 2005/0244939 and International Publication WO2007/103989. Therefore, in the present invention, these methods can be used in order to prepare 4R-IHOG from IPA and PA.

In addition, for the aldolase used in the condensation reaction, the enzymes disclosed in the specifications of WO 2003/091396 and US Patent Application Publication No. 2005/0244937 may be used. For example, the following enzymes are used. As mentioned above, the following enzyme is abbreviated as the aldolase, as long as it can form 4R-IHOG from IPA and PA.

EC 4.1.3.—: synthases/lyases that form carbon-carbon bonds utilizing oxo-acid substrates (such as indole-3-pyruvate) as the electrophile.

For example, such an enzyme includes the polypeptide described in EP 1045-029 (EC 4.1.3.16, 4-hydroxy-2-oxoglutarate glyoxylate-lyase also termed 4-hydroxy-2-oxoglutarate aldolase, 2-oxo-4-hydroxyglutarate aldolase or KHG aldolase), and the polypeptide 4-hydroxy-4-methyl-2-oxoglutarate aldolase (EC 4.1.3.17, also termed 4-hydroxy-4-methyl-2-oxoglutarate pyruvate-lyase or ProA aldolase).

In one embodiment, the contact of IPA and PA with the aldolase can be accomplished by allowing IPA and PA, and the aldolase extracted from an aldolase-producing microorganism (extracted enzyme) to coexist in the reaction solution. Examples of the aldolase-producing microorganism include microorganisms that naturally produce the aldolase and transformants that express the aldolase. Specifically examples of the extracted enzyme include a purified enzyme, a crude enzyme, an enzyme-containing fraction prepared from the above enzyme-producing microorganism, and a disrupted product of and a lysate of the above enzyme-producing microorganism.

In another embodiment, the contact of IPA and PA with the aldolase can be accomplished by allowing IPA and PA and the aldolase-producing microorganism to coexist in the reaction solution (e.g., culture medium).

IPA used for the preparation of 4R-IHOG is an unstable compound. Therefore, the condensation of IPA and PA may be carried out in the presence of a stabilizing factor for IPA. Examples of the stabilizing factor for IPA include superoxide dismutase (see, e.g., International Publication WO2009/028338) and mercaptoethanol (see, e.g., International Publication WO2009/028338). For example, the transformant expressing the superoxide dismutase is disclosed in International Publication WO2009/028338. Thus, such a transformant may be used in the method of the present invention.

Various conditions such as the reaction solution, the temperature, the pH value and the time period in the condensation reaction can be appropriately established as long as the objective reaction can progress. For example, the conditions for the condensation reaction may be the same as those described in the deamination reaction.

### (3) Amination Reaction

As used herein, the term “D-aminotransferase” refers to an enzyme capable of forming 2R,4R-Monatin by transferring the amino group in the D-amino acid to 4R-IHOG. Examples of the method for forming 2R,4R-Monatin by transferring the amino group in the D-amino acid to 4R-IHOG by the D-aminotransferase are disclosed in International Publication WO2004/053125. Therefore, these methods can be used in the present invention in order to prepare 2R,4R-Monatin from 4R-IHOG in the presence of the D-amino acid.

In addition, for the D-aminotransferase used in the amination reaction, the enzymes disclosed in the specifications of WO 2003/091396 and US Patent Application Publication No. 2005/0244937 may be used. For example, the following enzymes are used. As mentioned above, the following enzyme is abbreviated as the D-aminotransferase, as long as it can transfer the amino group of the D-amino acid to 4R-IHOG to form 2R,4R-Monatin.

EC 2.6.1.27: tryptophan aminotransferases

EC 1.4.1.19: tryptophan dehydrogenases

EC 1.4.99.1: D-amino acid dehydrogenases

EC 1.4.1.2-4: glutamate dehydrogenases

EC 1.4.1.20: phenylalanine dehydrogenase

EC 2.6.1.28: tryptophan-phenylpyruvate transaminases

EC 2.6.1.1: aspartate aminotransferase

EC 2.6.1.5: tyrosine (aromatic) aminotransferase

EC 2.6.1.—: aminotransferase family. For example, it includes D-tryptophan aminotransferase, or D-alanine aminotransferase.

In one embodiment, the contact of 4R-IHOG with the D-aminotransferase in the presence of the D-amino acid can be accomplished by allowing the 4R-IHOG and the D-aminotransferase extracted from a D-aminotransferase-producing microorganism (extracted enzyme) to coexist in the reaction solution containing the D-amino acid. Examples of the D-aminotransferase-producing microorganism include microorganisms that naturally produce the D-aminotransferase and transformants that express the D-aminotransferase. Specifically, examples of the extracted enzyme include a purified enzyme, a crude enzyme, an enzyme-containing fraction prepared from the above enzyme-producing microorganism, and a disrupted product of and a lysate of the above enzyme-producing microorganism.

In another embodiment, the contact of 4R-IHOG with the D-aminotransferase in the presence of the D-amino acid can be accomplished by allowing the 4R-IHOG and the D-aminotransferase-producing microorganism to coexist in the reaction solution (e.g., culture medium) containing the D-amino acid.

The kinds of the D-amino acid are not particularly limited as long as the amino group in the D-amino acid can be transferred to 4R-IHOG that is an objective substrate by the D-aminotransferase. Various D-amino acids such as D-α-amino acids are known as such a D-amino acid. Specifically, such a D-amino acid includes D-aspartic acid, D-alanine, D-lysine, D-arginine, D-histidine, D-glutamic acid, D-asparagine, D-glutamine, D-serine, D-threonine, D-tyrosine, D-cysteine, D-valine, D-leucine, D-isoleucine, D-proline, D-phenylalanine, D-methionine and D-tryptophan.

Various conditions such as the reaction solution, the temperature, the pH value and the time period in the amination reaction can be appropriately established as long as the objective reaction can progress. For example, the conditions for the amination reaction may be the same as those described in the deamination reaction. The reaction solution for the amination reaction may further contain pyridoxal phosphate (PLP) as a coenzyme.

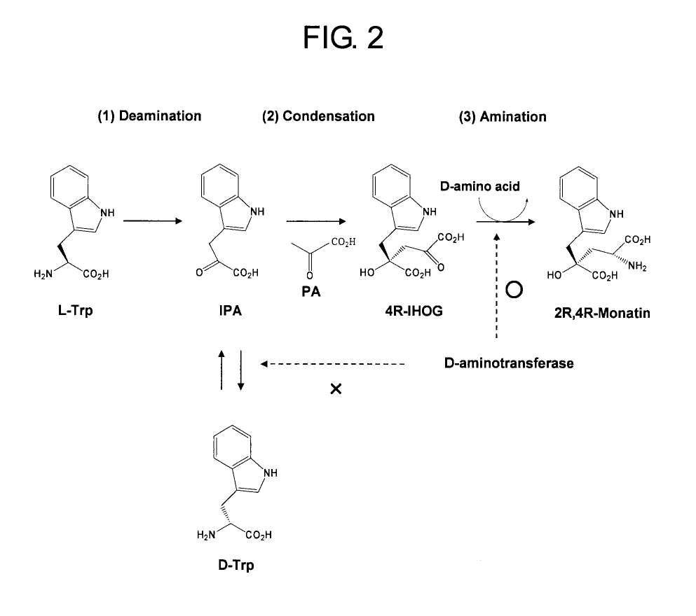

Preferably, the D-aminotransferase used for the amination reaction may be one having an ability to form 2R,4R-Monatin from 4R-IHOG in the presence of the D-amino acid and having no or low ability to form D-Trp from IPA (FIG. 2). A nature of such a D-aminotransferase can also be represented as a ratio of a 4R-IHOG amination activity to an IPA amination activity. Preferably a D-aminotransferase having the IPA amination activity that is lower than the 4R-IHOG amination activity, more preferably a D-aminotransferase having the IPA amination activity that may be 1/10 of the 4R-IHOG amination activity, still more preferably D-aminotransferase having the IPA amination activity that may be 1/100 or less of the 4R-IHOG amination activity, and particularly preferably the D-aminotransferase having no IPA amination activity can be used. By the use of such a D-aminotransferase, the 2R,4R-Monatin can be produced with a good yield because the formation of D-Trp from IPA is suppressed and the formation of 4R-IHOG from IPA and PA is promoted (FIG. 2).

The aforementioned D-aminotransferase can be a protein derived from a microorganism such as a bacterium, actinomycete or yeast. The classification of the microorganisms can be carried out by a classification method well-known in the art, e.g., a classification method used in the database of NCBI (National Center for Biotechnology Information) (http://www.ncbi.nlm.nih.gov/Taxonomy/Browser/wwwtax.cgi?id=91347). Examples of the microorganisms from which such a D-aminotransferase is derived include microorganisms belonging to genus Achromobacter, genus Agrobacterium, genus Bacillus, genus Coprococcus, genus Geobacillus, genus Halothiobacillus, genus Lactobacillus, genus Oceanibulbus, genus Paenibacillus, genus Rhodobacter, genus Robiginitalea, and genus Thiobacillus. Specifically, examples of such microorganisms include Achromobacter xylosoxidans, Agrobacterium radiobacter, Bacillus halodurans, Bacillus megaterium, Bacillus macerans, Bacillus proteiformans, Bhalodurans, Coprococcus comes, Geobacillus sp., Geobacillus toebii, Halothiobacillus neapolitanus, Lactobacillus salivarius, Oceanibulbus indolifex, Paenibacillus larvae, Rhodobacter sphaeroides, Robiginitalea biformata, and Thiobacillus denitrificans.

The aforementioned D-aminotransferase can be a naturally occurring protein or an artificial mutant protein. Such a D-aminotransferase can be screened from any D-aminotransferases expressed by the microorganisms such as the bacteria, the actinomycetes or the yeasts. Examples of the D-aminotransferase include proteins consisting of an amino acid sequence having a homology (e.g., similarity or identity) of 80% or more, preferably 90% or more, more preferably 95% or more, particularly 98 or more or 99% or more to an amino acid sequence represented by SEQ ID NO:2, SEQ ID NO:8, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84 or SEQ ID NO:86, and having a D-aminotransferase activity. Such a D-aminotransferase can also be obtained by a) introducing one or more amino acid mutations into any D-aminotransferase to produce D-aminotransferase mutants and b) selecting one retaining an ability to form 2R,4R-Monatin from 4R-IHOG in the presence of the D-amino acid and having no or low ability to form D-Trp from IPA among from the produced D-aminotransferase mutants. Examples of such a D-aminotransferase mutant may be a protein consisting of an amino acid sequence comprising a mutation (e.g., deletion, substitution, addition and insertion) of one or several amino acid residues in an amino acid sequence represented by SEQ ID NO:2, SEQ ID NO:8, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84 or SEQ ID NO:86, and having a D-aminotransferase activity. The mutation of one or several amino acid residue may be introduced into one region in the amino acid sequence, or may be introduced into plural different regions in the amino acid sequence. The term “one or several” indicate a range in which a three dimensional structure and the activity of the protein are not largely impaired. The term “one or several” in the case of the protein denote, for example, 1 to 100, preferably 1 to 80, more preferably 1 to 50, 1 to 30, 1 to 20, 1 to 10 or 1 to 5. Such mutation may be attributed to naturally occurring mutation (mutant or variant) based on individual difference, species difference and the like of the microorganism carrying a gene encoding the D-aminotransferase.

Examples of such a D-aminotransferase mutant also may be a protein comprising a mutation of one or more (e.g., 1, 2, 3, 4, 5, 6, 7 or 8) amino acid residues selected from the group consisting of the amino acid residues at positions 87, 100, 117, 145, 157, 240, 243 and 244 in the amino acid sequence represented by SEQ ID NO:2, or comprising a mutation of one or more amino acid residues selected from the group consisting of the amino acid residues that are present at the positions corresponding to the aforementioned positions on SEQ ID NO:2 in an amino acid sequence represented by SEQ ID NO:8, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84 or SEQ ID NO:86, and having the D-aminotransferase activity as mentioned above. The amino acid residues in SEQ ID NO:8 and the like that are present at the positions corresponding to the aforementioned positions on SEQ ID NO:2 can be determined by alignment comparison of amino acid sequences. For example, the mutation of the amino acid residue in an amino acid sequence represented by SEQ ID NO:2 and the like may be a substitution of the amino acid residue selected from the group consisting of the followings:

i) the substitution of histidine at position 87 with arginine;
ii) the substitution of asparagine at position 100 with threonine;
iii) the substitution of lysine at position 117 with arginine or glutamine;
iv) the substitution of isoleucine at position 145 with valine;
v) the substitution of lysine at position 157 with arginine, glutamine or threonine;
vi) the substitution of serine at position 240 with threonine;
vii) the substitution of serine at position 243 with asparagine; and
viii) the substitution of serine at position 244 with lysine.

The mutation of the amino acid residues may comprise combinations of one or more of the substitutions i) to viii) (e.g., the substitution of serine at position 243 with asparagine and the substitution of serine at position 244 with lysine).

The D-aminotransferase mutant containing the mutations of the amino acid residues at the aforementioned positions in the amino acid sequence represented by SEQ ID NO:2, SEQ ID NO:8, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84 or SEQ ID NO:86 includes (I) proteins in which the amino acid residues have been mutated at the aforementioned positions in the amino acid sequence represented by SEQ ID NO:2, SEQ ID NO:8, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84 or SEQ ID NO:86, and (II) those which consist of an amino acid sequence having high homology (e.g., similarity, identity) to the amino acid sequence in which the amino acid residues have been mutated at the aforementioned positions in the amino acid sequence represented by SEQ ID NO:2, SEQ ID NO:8, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84 or SEQ ID NO:86 (hereinafter referred to as a mutant amino acid sequence as needed), and have the D-aminotransferase activity. The term “D-aminotransferase activity” refers to an activity of transferring the amino group in the D-amino acid to 4R-IHOG that is the objective substrate for forming 2R,4R Monatin that is an objective compound having the amino group. Specifically, the D-aminotransferase includes a protein consisting of an amino acid sequence showing 80% or more, preferably 90% or more, more preferably 95% or more and particularly preferably 98% or more or 99% or more homology (e.g., similarity, identity) to the mutant amino acid sequence (the mutations of one or more amino acid residues at the aforementioned positions are conserved), and having the D-aminotransferase activity.

The homology of the amino acid sequences and nucleotide sequences can be determined using algorithm BLAST by Karlin and Altschul (Pro. Natl. Acad. Sci. USA, 90, 5873 (1993)) or FASTA by Pearson (Methods Enzymol., 183, 63 (1990)). Programs referred to as BLASTP and BLASTN (see http://www.ncbi.nlm.nih.gov) have been developed based on this algorithm BLAST. Thus, the homology of the amino acid sequences and the nucleotide sequences may be calculated using these programs with default setting. A numerical value obtained when matching count is calculated as a percentage by using GENETYX Ver. 7.0.9 that is software from GENETYX Corporation and using full length polypeptide chains encoded in ORF with setting of Unit Size to Compare=2 may be used as the homology of the amino acid sequences. The lowest value among the values derived from these calculations may be employed as the homology of the amino acid sequences and the nucleotide sequences.

The D-aminotransferase mutant may be a protein consisting of an amino acid sequence comprising mutation (e.g., deletion, substitution, addition and insertion) of one or several amino acid residues in the mutant amino acid sequence (the mutations of one or more amino acid residues at the aforementioned positions are conserved), and having the D-aminotransferase activity. The mutation of one or several amino acid residues may be introduced into one region or multiple different regions in the amino acid sequence. The term “one or several amino acid residues” indicate a range in which a three dimensional structure and the activity of the protein are not largely impaired. The term “one or several amino acid residues” in the case of the protein denote, for example, 1 to 100, preferably 1 to 80, more preferably 1 to 50, 1 to 30, 1 to 20, 1 to 10 or 1 to 5 amino acid residues. Such mutation may be attributed to naturally occurring mutation (mutant or variant) based on individual difference, species difference and the like of the microorganism carrying a gene encoding the D-aminotransferase. The D-aminotransferase mutant may comprise a tag for purification such as a histidine tag.

A position of the amino acid residue to be mutated in the amino acid sequence is apparent to those skilled in the art. Specifically, a person skilled in the art can recognize the correlation between structure and function by 1) comparing the amino acid sequences of the multiple proteins having the same kind of activity (e.g., the amino acid sequence represented by SEQ ID NO:2, and amino acid sequences of other L-aminotransferase), 2) clarifying relatively conserved regions and relatively non-conserved regions, and then 3) predicting a region capable of playing an important role for its function and a region incapable of playing the important role for its function from the relatively conserved regions and the relatively non-conserved regions, respectively. Therefore, a person skilled in the art can specify the position of the amino acid residue to be mutated in the amino acid sequence of the L-aminotransferase.

When an amino acid residue is mutated by the substitution in the mutant amino acid sequence (the mutations of one or more amino acid residues at the aforementioned positions are conserved), the substitution of the amino acid residue may be conservative substitution. As used herein, the term “conservative substitution” means that a certain amino acid residue is substituted with an amino acid residue having an analogous side chain. Families of the amino acid residues having the analogous side chain are well-known in the art. Examples of such families include an amino acid having a basic side chain (e.g., lysine, arginine or histidine), an amino acid having an acidic side chain (e.g., aspartic acid or glutamic acid), an amino acid having a non-charged polar side chain (e.g., asparagine, glutamine, serine, threonine, tyrosine or cysteine), an amino acid having a non-polar side chain (e.g., glycine, alanine, valine, leucine, isoleucine, proline, phenylalanine, methionine or tryptophan), an amino acid having a β-position branched side chain (e.g., threonine, valine or isoleucine), an amino acid having an aromatic side chain (e.g., tyrosine, phenylalanine, tryptophan or histidine), an amino acid having a hydroxyl group (e.g., alcoholic or phenolic)-containing side chain (e.g., serine, threonine or tyrosine), and an amino acid having a sulfur-containing side chain (e.g., cysteine or methionine). Preferably, the conservative substitution of the amino acids may be the substitution between aspartic acid and glutamic acid, the substitution among arginine, lysine and histidine, the substitution between tryptophan and phenylalanine, the substitution between phenylalanine and valine, the substitution among leucine, isoleucine and alanine, and the substitution between glycine and alanine.

The D-aminotransferase mutant may be a protein encoded by DNA that hybridizes under a stringent condition with a nucleotide sequence complementary to a nucleotide sequence represented by SEQ ID NO:1, SEQ ID NO:7, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83 or SEQ ID NO:85, and having the D-aminotransferase activity. The “stringent condition” refers to a condition where a so-called specific hybrid is formed whereas no non-specific hybrid is formed. Although it is difficult to clearly quantify this condition, one example of this condition is the condition where a pair of polynucleotides with high homology (e.g., identity), for example, a pair of polynucleotides having the homology of 80% or more, preferably 90% or more, more preferably 95% or more, and particularly preferably 98% or more are hybridized whereas a pair of polynucleotides with lower homology than that are not hybridized. Specifically, such a condition includes hybridization in 6×SSC (sodium chloride/sodium citrate) at about 45° C. followed by one or two or more washings in 0.2×SSC and 0.1% SDS at 50 to 65° C.

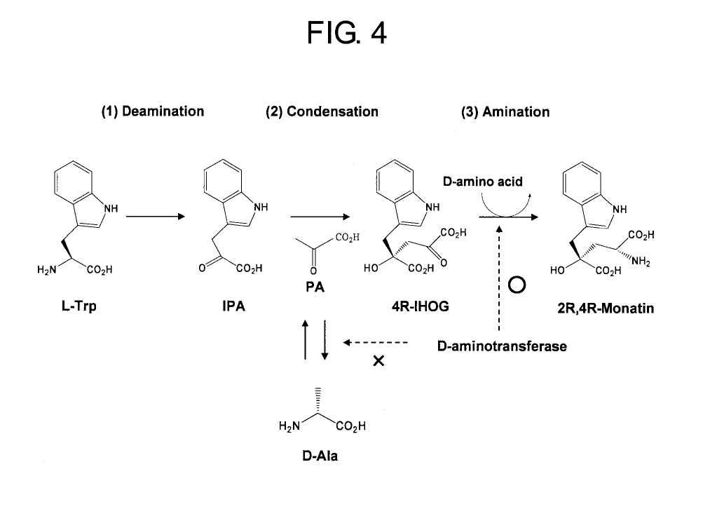

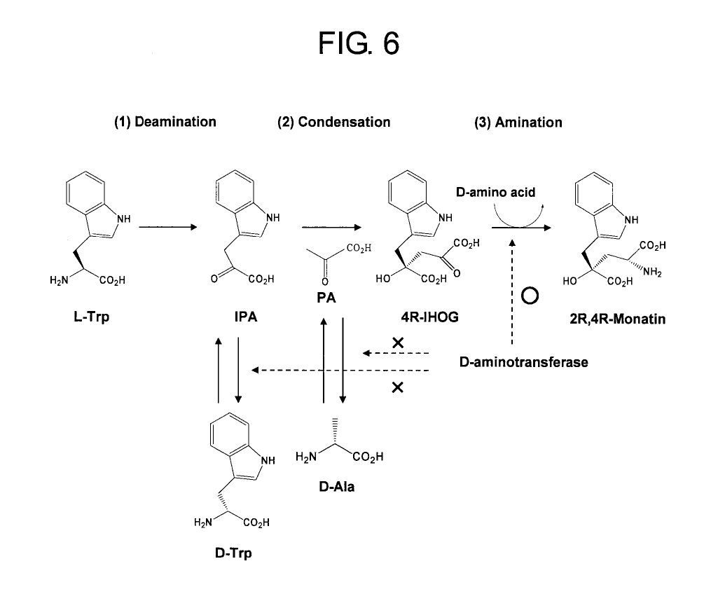

The D-aminotransferase used for the amination reaction may be one having an ability to form 2R,4R-Monatin from 4R-IHOG in the presence of the D-amino acid, and having no or low ability to form D-alanine (D-Ala) from PA (FIGS. 4 and 6). The nature of such a D-aminotransferase can also be represented by the ratio of the 4R-IHOG amination activity to the PA amination activity. Preferably the D-aminotransferase having the PA amination activity that is lower than the 4R-IHOG amination activity, more preferably the D-aminotransferase having the PA amination activity that may be 1/10 of the 4R-IHOG amination activity, still more preferably D-aminotransferase having the PA amination activity that may be 1/100 or less of the 4R-IHOG amination activity, and particularly preferably the D-aminotransferase having no PA amination activity can be used. By the use of such a D-aminotransferase, 2R,4R-Monatin can be produced with a good yield because the formation of D-Ala from PA is suppressed and the formation of 4R-IHOG from IPA and PA is promoted (FIGS. 4 and 6). Such a D-aminotransferase can be obtained in the same manner as in the case of the aforementioned D-aminotransferase having an ability to form 2R,4R-Monatin from 4R-IHOG in the presence of the D-amino acid, and having no or low ability to form D-Trp from IPA. Preferably, the D-aminotransferase used for the amination reaction may also be one having an ability to form 2R,4R-Monatin from 4R-IHOG in the presence of the D-amino acid, having no or low ability to form D-Trp from IPA, and having no or low ability to form D-alanine (D-Ala) from PA.

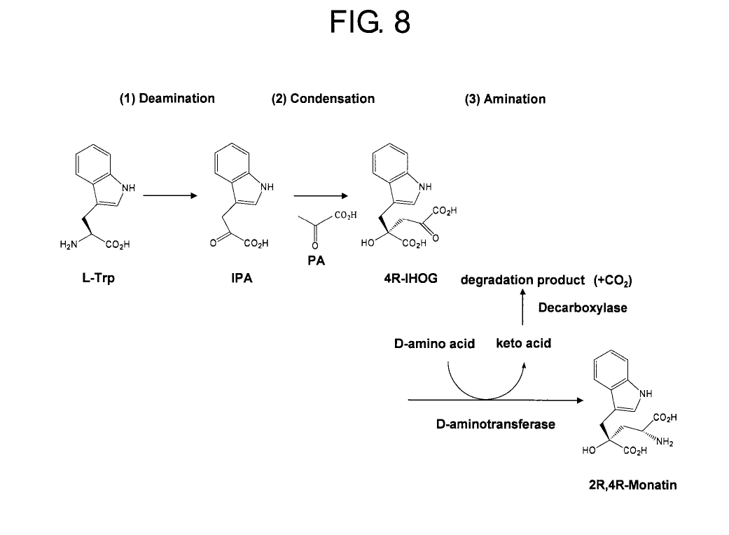

In a preferred embodiment, the production method of the present invention further comprises contacting a keto acid (R—COCOOH) formed from the D-amino acid (e.g., D-α-amino acid) by action of the D-aminotransferase with a decarboxylase to degrade the keto acid (FIG. 8). By promoting the degradation of the keto acid formed from the D-amino acid by an amino group transfer reaction, it is possible to shift the equilibrium of the reaction to form 2R,4R-Monatin from 4R-IHOG so that 2R,4R-Monatin is formed in a larger amount (FIG. 8).

The decarboxylase used in the present invention is an enzyme that catalyzes a decarboxylation reaction of the keto acid. The decarboxylation reaction by the decarboxylase may be irreversible. Various enzymes are known as the decarboxylase used for the irreversible decarboxylation reaction of the keto acid, and examples thereof include an oxaloacetate decarboxylase derived from Pseudomonas stutzeri (Arch Biochem Biophys., 365, 17-24, 1999) and a pyruvate decarboxylase derived from Zymomonas mobilis (Applied Microbiology and Biotechnology, 17, 152-157, 1983).

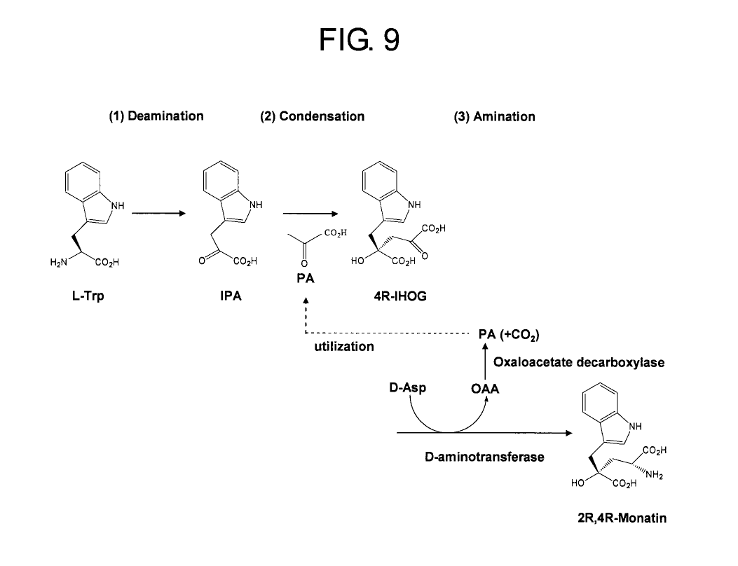

In a particularly preferred embodiment, the production method of the present invention comprises contacting oxaloacetate (OAA) formed from D-aspartic acid (D-Asp) by action of the D-aminotransferase with the oxaloacetate decarboxylase to form the pyruvate (PA) (FIG. 9). By promoting the irreversible formation of PA from OAA, it is possible to shift the equilibrium of the reaction to form 2R,4R-Monatin from 4R-IHOG so that 2R,4R-Monatin is formed in a larger amount (FIG. 9). When D-Asp is used as the D-amino acid that is one of the substrates in the amination reaction, the D-aminotransferase may have the higher substrate specificity for D-Asp than the substrate specificity for D-Trp or D-Ala, or the substrate specificity for D-Trp and D-Ala (FIGS. 2, 4 and 6). Considering reversibility of the reaction, when D-aminotransferase having such a nature is used, the reaction to from 2R,4R-Monatin from 4R-IHOG is thought to progress more easily than the reaction to form D-Trp from IPA and/or the reaction to form D-Ala from PA.

The oxaloacetate decarboxylase used in the present invention is an enzyme that catalyzes the decarboxylation reaction of OAA to form PA. The decarboxylation reaction by the oxaloacetate decarboxylase can be irreversible. Various enzymes are known as the oxaloacetate decarboxylase used for the irreversible decarboxylation reaction of OAA. Examples of such an oxaloacetate decarboxylase include the oxaloacetate decarboxylase derived from Pseudomonas stutzeri (Arch Biochem Biophys., 365, 17-24, 1999), the oxaloacetate decarboxylase derived from Klebsiella aerogenes (FEBS Lett., 141, 59-62, 1982), and the oxaloacetate decarboxylase derived from Sulfolobus solfataricus (Biochim Biophys Acta., 957, 301-311, 1988).

When the decarboxylase is used in the production of 2R,4R-Monatin from 4R-IHOG, the contact of the keto acid formed from the D-amino acid with the decarboxylase can be accomplished by allowing the keto acid and the decarboxylase extracted from a decarboxylase-producing microorganism (extracted enzyme) or the decarboxylase-producing microorganism to coexist in the reaction solution (e.g., culture medium). Examples of the decarboxylase-producing microorganism include microorganisms that naturally produce the decarboxylase and transformants that express the decarboxylase. Examples of the extracted enzyme include a purified enzyme, a crude enzyme, an enzyme-containing fraction prepared from the above enzyme-producing microorganism, and a disrupted product of and a lysate of the above enzyme-producing microorganism.

When both the D-aminotransferase and the decarboxylase are used in the production of 2R,4R-Monatin from 4R-IHOG, the D-aminotransferase and the decarboxylase may be provided in the reaction solution in the following manner:

D-aminotransferase (extracted enzyme) and decarboxylase (extracted enzyme);

D-aminotransferase-producing microorganism and decarboxylase (extracted enzyme);

D-aminotransferase (extracted enzyme) and decarboxylase-producing microorganism;

D-aminotransferase-producing microorganism and decarboxylase-producing microorganism; and

D-aminotransferase- and decarboxylase-producing microorganisms.

Preferably, the D-aminotransferase- and decarboxylase-producing microorganism may be a transformant. Such a transformant can be made by i) introducing an expression vector of the D-aminotransferase into the decarboxylase-producing microorganism, ii) introducing an expression vector of the decarboxylase into the D-aminotransferase-producing microorganism, (iii) introducing a first expression vector of the D-aminotransferase and a second expression vector of the decarboxylase into a host microorganism, and (iv) introducing an expression vector of the D-aminotransferase and the decarboxylase into the host microorganism. Examples of the expression vector of the D-aminotransferase and the decarboxylase include i′) an expression vector containing a first expression unit composed of a first polynucleotide encoding the D-aminotransferase and a first promoter operatively linked to the first polynucleotide, and a second expression unit composed of a second polynucleotide encoding the decarboxylase and a second promoter operatively linked to the second polynucleotide; and ii′) an expression vector containing a first polynucleotide encoding the D-aminotransferase, a second polynucleotide encoding the decarboxylase and a promoter operatively linked to the first polynucleotide and the second polynucleotide (vector capable of expressing polycistronic mRNA). The first polynucleotide encoding the D-aminotransferase may be located upstream or downstream the second polynucleotide encoding the decarboxylase.

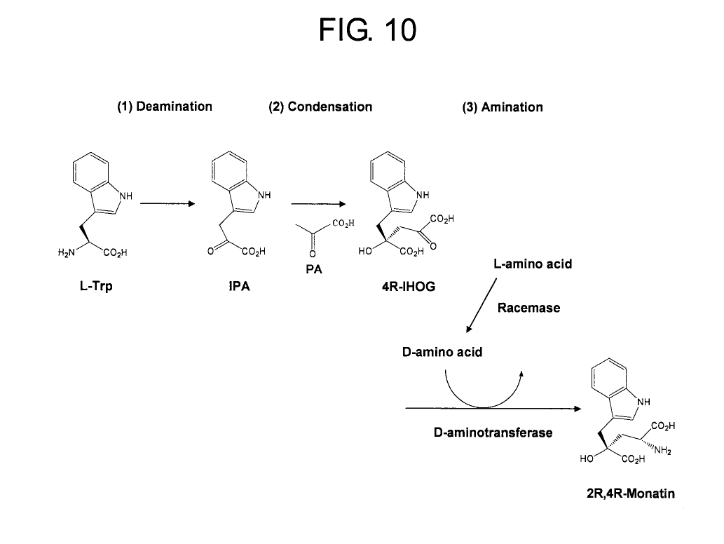

The production method of the present invention may further comprise contacting an L-amino acid with a racemase to form the D-amino acid (FIG. 10). The racemase used in the present invention is an enzyme to convert the L-amino acid to the D-amino acid. Examples of the method for forming the D-amino acid from the L-amino acid by the racemase are disclosed in Kuniki Kino et al., Synthesis of DL-tryptophan by modified broad specificity amino acid racemase from Pseudomonas putida IFO 12996. Applied Microbiology and Biotechnology (2007), 73 (6), 1299-1305, and Tohru Yoshimura et al., Amino acid racemases: Functions and mechanisms. Journal of Bioscience and Bioengineering (2003), 96 (2), 103-109. Therefore, these methods can be used for preparing the D-amino acid from the L-amino acid in the present invention.

Various L-amino acids such as L-α-amino acids are known as such an L-amino acid. Specifically, such an L-amino acid includes L-aspartic acid, L-alanine, L-lysine, L-arginine, L-histidine, L-glutamic acid, L-asparagine, L-glutamine, L-serine, L-threonine, L-tyrosine, L-cysteine, L-valine, L-leucine, L-isoleucine, L-proline, L-phenylalanine, L-methionine and L-tryptophan. L-Asp is preferable as the L-amino acid because D-Asp is preferable as the D-amino acid used for the amination reaction.

In one embodiment, the contact of the L-amino acid with the racemase can be accomplished by allowing the L-amino acid and the racemase extracted from a racemase-producing microorganism (extracted enzyme) to coexist in the reaction solution. The racemase-producing microorganism includes microorganisms that naturally produce the racemase and transformants that express the racemase. Specifically, examples of the extracted enzyme include a purified enzyme, a crude enzyme, an enzyme-containing fraction prepared from the above enzyme-producing microorganism, and a disrupted product of and a lysate of the above enzyme-producing microorganism.

In another embodiment, the contact of the L-amino acid with the racemase can be accomplished by allowing the L-amino acid and the racemase-producing microorganism to coexist in the reaction solution (e.g., culture medium).

When both the D-aminotransferase and the racemase are used in the production of 2R,4R-Monatin from 4R-IHOG, the D-aminotransferase and the racemase may be provided in the reaction solution in the same manner as in the aforementioned case of the D-aminotransferase and the decarboxylase.

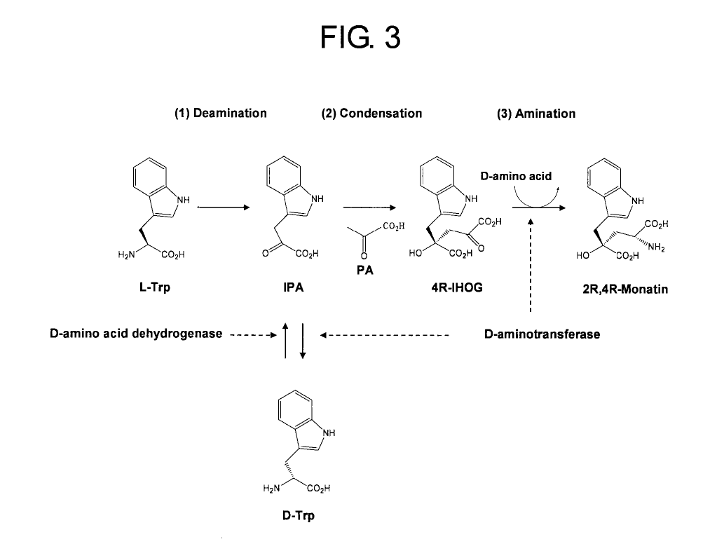

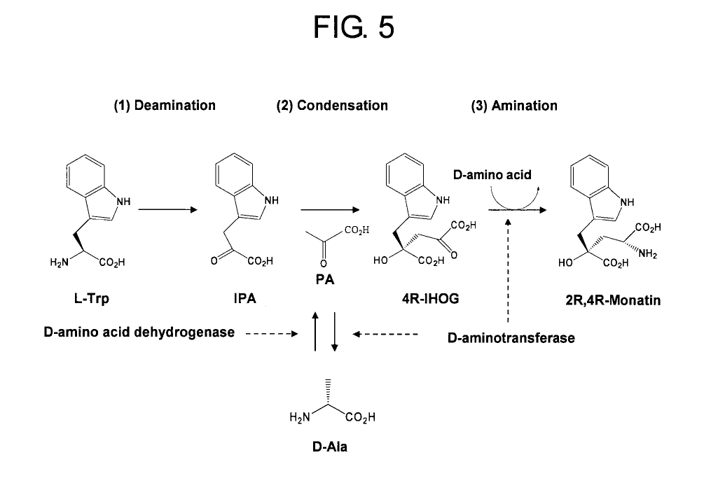

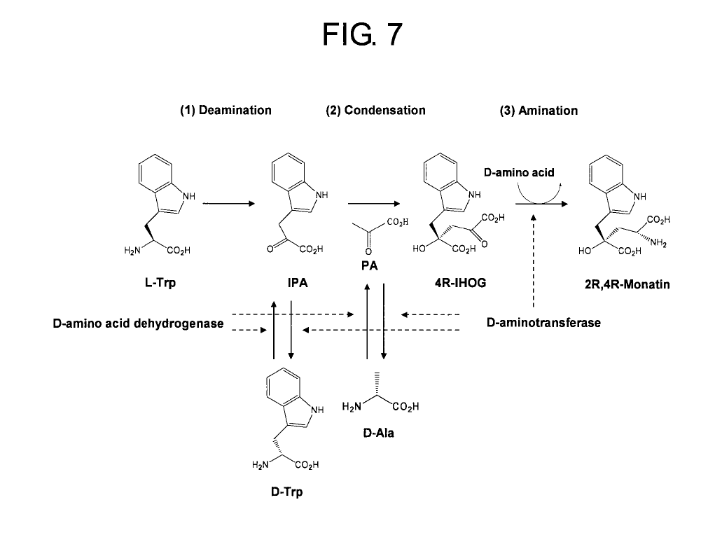

The production method of the present invention may comprise allowing a D-amino acid dehydrogenase to exist in the reactor in order to convert again D-Trp produced as the byproduct during the reaction into IPA (FIGS. 3, 5 and 7). The D-amino acid dehydrogenase used in the present invention is an enzyme to convert the D-amino acid into a corresponding keto acid. Examples of the D-amino acid dehydrogenase include the D-amino acid dehydrogenase using NAD(P) as a coenzyme, which is disclosed in Kavitha Vedha-Peters et al., Creation of a Broad-Range and Highly Stereoselective D-Amino Acid Dehydrogenase for the One-Step Synthesis of D-Amino Acids. Journal of the American Chemical Society (2006), 128(33), 10923-10929, and the D-amino acid dehydrogenase (E.C. 1.4.5.1) using quinone as the coenzyme, which is disclosed in M. Tanigawa et al., D-amino acid dehydrogenase from Helicobacter pylori NCTC11637, Amino Acid (2010) 38: 247-255.

When a transformant that expresses the objective enzyme (e.g., deamination enzyme, aldolase, D-aminotransferase, decarboxylase, racemase) is used as the objective enzyme-producing microorganism, this transformant can be made by making an expression vector of the objective enzyme, and then introducing this expression vector into a host. For example, the transformant that expresses the D-aminotransferase mutant of the present invention can be obtained by making the expression vector incorporating DNA encoding the D-aminotransferase mutant of the present invention, and introducing it into an appropriate host.

For example, various prokaryotic cells including bacteria belonging to genus Escherichia such as Escherichia coli, genus Corynebacterium and Bacillus subtilis, and various eukaryotic cells including Saccharomyces cerevisiae, Pichia stipitis and Aspergillus oryzae can be used as the host for expressing the objective enzyme.

The hosts to be transformed are as described above. Describing Escherichia coli in detail, the host can be selected from Escherichia coli K12 strain subspecies, Escherichia coli JM109, DH5α, HB101, BL21 (DE3) strains and the like. Methods for performing the transformation and methods for selecting the transformant are described in Molecular Cloning: A Laboratory Manual, 3rd edition, Cold Spring Harbor press (2001/01/15) and the like. A method for making transformed Escherichia coli and producing a certain enzyme by the use thereof will be specifically described below as one example.

As a promoter for expressing DNA encoding the objective enzyme, the promoter typically used for producing a heterogeneous protein in E. coli can be used, and includes potent promoters such as T7 promoter, lac promoter, trp promoter, trc promoter, tac promoter, PR and PL promoters of lambda phage, and T5 promoter. As the vector, pUC19, pUC18, pBR322, pHSG299, pHSG298, pHSG399, pHSG398, RSF1010, pACYC177, pACYC184, pMW119, pMW118, pMW219, pMW218, pQE30 and derivatives thereof, and the like may be used. The vectors of phage DNA may also be utilized as the other vectors. Further, the expression vector containing the promoter and capable of expressing the inserted DNA sequence may be used.

A terminator that is a transcription termination sequence may be ligated to downstream of an objective enzyme gene. Examples of such a terminator include T7 terminator, fd phage terminator, T4 terminator, a terminator of a tetracycline resistant gene, and a terminator of an E. coli trpA gene.

So-called multiple copy types are preferable as the vector for introducing the objective enzyme gene into E. coli, and include plasmids having a replication origin derived from ColE1, such as pUC type plasmids, pBR322 type plasmids or derivatives thereof. Here, the “derivatives” means those in which modification is given to the plasmids by substitution, deletion, insertion, addition and/or inversion of nucleotides. The “modification” as referred to here also includes the modification by mutagenic treatments by mutagenic agents and UV irradiation, or natural mutation, or the like.

For selecting the transformant, it is preferable that the vector has a marker such as an ampicillin resistant gene. As such a plasmid, the expression vectors carrying the strong promoter are commercially available (e.g., pUC types (supplied from TAKARA BIO Inc.), pPROK types (supplied from Clontech), pKK233-2 (supplied from Clontech)).

The objective enzyme is expressed by transforming E. coli with the obtained expression vector and culturing this E. coli.

A medium such as M9-casamino acid medium and LB medium typically used for culturing E. coli may be used as the medium. Culture conditions and production induction conditions are appropriately selected depending on types of the marker and the promoter in the used vector, the host bacterium and the like.

The following methods and the like are available for recovering the objective enzyme. The objective enzyme can be obtained as a disrupted product or a lysate by collecting the objective enzyme-producing microorganism followed by disrupting (e.g., sonication, homogenization) or lysing (e.g., lysozyme treatment) the microbial cells. Also, the purified enzyme, the crude enzyme or the enzyme-containing fraction can be obtained by subjecting such a disrupted product or lysate to techniques such as extraction, precipitation, filtration and column chromatography.

The 2R,4R-Monatin obtained by the production method of the present invention can be isolated and purified by combining known separation and purification procedures such as concentration, reduced pressure concentration, solvent extraction, crystallization, recrystallization, solvent transfer, a treatment with activated charcoal, and treatments with chromatography and the like using ion exchange resin or synthetic adsorption resin, as needed. The compound used as the raw material in the production method of the present invention may be added in a salt form to the reaction system, unless otherwise specified. The salt of 2R,4R-Monatin produced in the present invention can be produced, for example, by adding an inorganic acid or an organic acid to 2R,4R-Monatin according to the method publicly known per se. The 2R,4R-Monatin and the salt thereof may be hydrate, and both hydrate and non-hydrate are included in the scope of the present invention. The salt includes various salts such as sodium salts, potassium salts, ammonium salts, magnesium salts, and calcium salts.

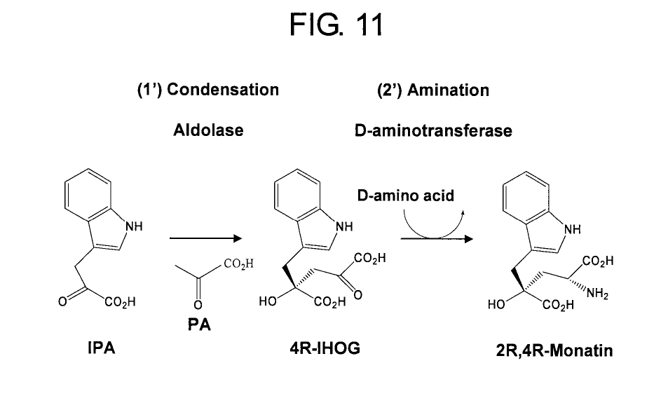

The present invention also provides a method for producing 2R,4R-Monatin or a salt thereof, comprising the following two steps carried out in one reactor (FIG. 11).

(1′) Contacting indole-3-pyruvate and pyruvate with an aldolase to form 4R-IHOG and
(2′) Contacting 4R-IHOG with a D-aminotransferase in the presence of a D-amino acid to form 2R,4R-Monatin

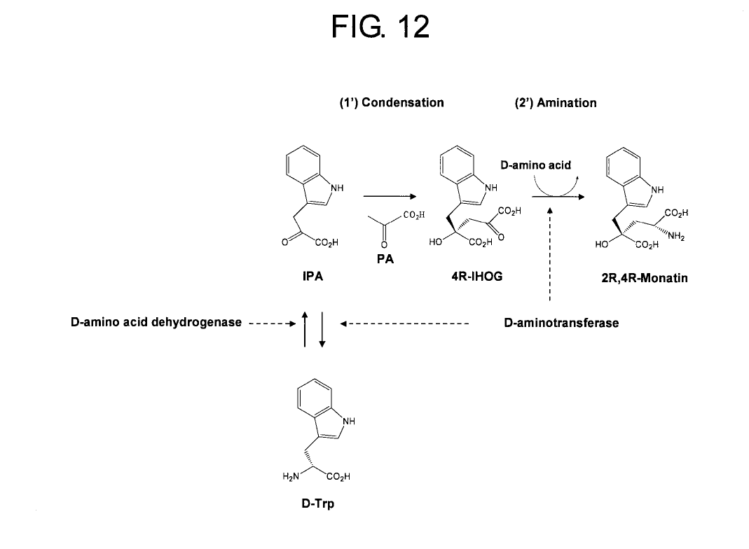

This production method can be carried out in the same manner as in the steps (2) and (3) in the aforementioned production method of the present invention. For example, the production method may further comprise allowing a D-amino acid dehydrogenase to exist in the reaction solution (FIG. 12). This production method may further comprise the same step as the step (1) in the production method of the present invention.

The present invention will be described in detail by the following Examples, but the present invention is not limited by these Examples.

### EXAMPLES

### Example 1

### Construction of Strain that Expresses DAT Derived from Bacillus macerans AJ1617 Strain and Measurement of its Activity

### 1) Construction of BMDAT-Expressing Strain

PCR amplification was carried out using a plasmid in which Bacillus macerans AJ1617 strain-derived dat gene (BMDAT gene) described in International Publication WO2004/053125 had been inserted as a template. Hereinafter, an S244K mutant enzyme and an S243N/S244K mutant enzyme are referred to as BMDAT22 and BMDAT80, respectively. The primer BmDAT-Nde-f (5′-ggatgaacggcatATGGCATATTCATTATGGAATGATC-3′: SEQ ID NO:3) and the primer BmDAT-delNde-r (5′-ttcaaagttttcataCgcacgttcacccgc-3′: SEQ ID NO:4) were used. Likewise, the primer BmDAT-delNde-f (5′-gcgggtgaacgtgcGtatgaaaactttgaa-3′: SEQ ID NO:5 and the primer BmDAT-Xho-r (5′-CAAGGTTCTTctcgagTTTGGTATTCATTGAAAGTGGTAATTTCGC-3′: SEQ ID NO:6) were also used for the PCR amplification. PCR amplification was carried out using two DNA fragments obtained in this way as the templates. The primer BmDAT-Nde-f and the primer BmDAT-Xho-r were used as the primers. All of the PCR amplifications were carried out using KOD-Plus-ver.2 (Toyobo). The resulting DNA fragments include BMDAT genes in which NdeI recognition site was deleted.

The condition for the PCR amplification was as follows:

| | |
|---|---|
| 1 cycle | 94° C., 2 min |
| 25 cycles | 98° C., 10 sec |
| | 55° C., 10 sec |
| | 68° C., 1 min |
| 1 cycle | 68° C., 1 min |

This DNA fragment was treated with restriction enzymes NdeI and XhoI, and then ligated to pET-22b (Novagen) likewise treated with the restriction enzymes NdeI and XhoI. E. coli JM109 was transformed with this ligation solution, an objective plasmid was extracted from ampicillin resistant colonies, and this plasmid was designated as pET22-BMDAT-His(C). E. coli BL21 (DE3) was transformed using this plasmid to obtain pET22-BMDAT-His(C)/E. coli BL21 (DE3). In this expression strain, BMDAT in which His-tag was added to the C terminus is expressed.

Likewise, a BMDAT22-expressing strain and a BMDAT80-expressing strain were constructed.

### 2) Purification of BMDAT

Microbial cells of the expression strain, pET22-BMDAT-His(C)/E. coli BL21 (DE3) grown on an LB-amp (100 mg/L) plate were inoculated to 160 mL of Overnight Express Instant TB Medium (Merck) containing 100 mg/L of ampicillin, and cultured with shaking at 30° C. for 16 hours using a Sakaguchi flask.

After the termination of the cultivation, microbial cells were collected from the resulting cultured medium by centrifugation, washed with and suspended in 20 mM Tris-HCl (pH 7.6), 100 mM NaCl and 20 mM imidazole, and disrupted by sonication. Microbial cell debris was removed from the disrupted solution by centrifugation, and the resulting supernatant was used as a soluble fraction.

The obtained soluble fraction was applied onto a His-tag protein purification column His Prep FF 16/10 (supplied from Pharmacia (GE Health Care Bioscience), CV=20 mL) equilibrated with 20 mM Tris-HCl (pH 7.6), 100 mM NaCl and 20 mM imidazole, and adsorbed to the carrier. Proteins that had not been adsorbed to the carrier (unadsorbed proteins) were washed out with 20 mM Tris-HCl (pH 7.6), 100 mM NaCl and 20 mM imidazole, and subsequently the adsorbed proteins were eluted by linearly changing the concentration of imidazole from 20 mM to 250 mM at a flow rate of 3 mL/minute.

Fractions containing BMDAT-His(C) were combined and concentrated using Amicon Ultra-15 10K (Millipore). The concentrated solution was diluted with 20 mM Tris-HCl (pH 7.6) to use as a BMDAT-His(C) solution.

Likewise, BMDAT22-His(C) and BMDAT80-His(C) were purified.

### 3) Measurement of DAT Activity

The BMDAT-His(C) solution, the BMDAT22-His(C) solution and the BMDAT80-His(C) solution obtained as above were used as enzyme sources. The enzyme was diluted with 20 mM Tris-HCl (pH 7.6) and 0.01% BSA. The reaction condition was as follows.

### Activity for D-Ala-αKG (α-Ketoglutaric Acid)

The activity was measured in 100 mM D-Ala, 10 mM αKG-2Na, 100 mM Tris-HCl (pH 8.0), 50 μM PLP, 0.25 mM NADH and 10 U/mL of LDH at 25° C. The reaction was carried out on a scale of 1 mL for 10 minutes, and the activity was calculated from the reduction of the absorbance measured at 340 nm. D-Lactate dehydrogenase from Leuconostoc mesenteroides (Oriental Yeast) was used as LDH.

### Activity for D-Ala-(±)-IHOG

The activity was measured in 100 mM D-Ala, 10 mM (±)-IHOG (defined the same as 4R/4S-IHOG), 100 mM Tris-HCl (pH 8.0), and 50 μM PLP at 25° C. The reaction was carried out on a scale of 0.2 mL for 15 minutes, and the formed 2R,4R-Monatin (RR) and 2R,4S-Monatin (RS) were quantified by UPLC analysis to calculate the activity. The reaction was stopped with a 200 mM sodium citrate solution (pH 4.5).

The condition for the UPLC analysis was as follows.

### Column: ACQUITY UPLC HSS T3 Column, 2.1×50 mm, 1.8 μm

### (Waters)

### Injection: 5

Column temperature: 40° C.
Detection wavelength: 210 nm
Flow rate: 0.5 mL/minute
Mobile phase: 20 mM KH₂PO₄/CH₃CN=96/4

### Activity for D-Ala-IPA

The activity was measured in 100 mM D-Ala, 10 mM IPA, 100 mM Tris-HCl (pH 8.0) and 50 μM PLP at 25° C. After preparing the reaction solution, the pH value was adjusted to pH 8.0 using 1 M NaOH. The reaction was carried out on a scale of 0.2 mL for 15 minutes, and formed Trp was quantified by the UPLC analysis to calculate the activity. The reaction was stopped with the 200 mM sodium citrate solution (pH 4.5).

The condition for the UPLC analysis was as above.

Activity for D-Ala-(±)-MHOG (4-hydroxy-4-methyl-2-oxo glutarate)

The activity was measured in 100 mM D-Ala, 10 mM (±)-MHOG (defined the same as 4R/4S-MHOG), 100 mM Tris-HCl (pH 8.0), 50 μM PLP, 0.25 mM NADH and 10 U/mL of LDH at 25° C. The reaction was carried out on a scale of 1 mL for 10 minutes, and the activity was calculated from the reduction of the absorbance measured at 340 nm. D-Lactate dehydrogenase from Leuconostoc mesenteroides (Oriental Yeast) was used as LDH.

Obtained results are shown in Table 1 (Unit is U/mg).

TABLE 1
Activity of DAT derived from AJ1617

Amino group receptor

| | D-Ala- | D-Ala-(±)- | D-Ala- | D-Ala-(±)- |
|---|---|---|---|---|
| | αKG | IHOG | IPA | MHOG |
| BMDAT | 57.8 | RR: 0.245 | 3.04 | 1.61 |
| | | RS: 0.271 | | |
| BMDAT22 | 17.8 | RR: 0.0957 | 0.115 | 2.51 |
| | | RS: 0.0272 | | |
| BMDAT80 | 7.45 | RR: 0.0837 | 0.645 | 4.50 |
| | | RS: 0.00136 | | |

RR: Formation of 2R,4R-Monatin
RS: Formation of 2R,4S-Monatin

### CONCLUSIONS

1) As described above, it was shown that the D-aminotransferase mutant of the present invention had remarkably reduced activity for IPA while having an ability to form 2R,4R-Monatin from 4R-IHOG in the presence of a D-amino acid.
2) The deamination reaction by the deamination enzyme and the condensation reaction by the aldolase is known as described above. Therefore, 2R,4R-Monatin can be produced from L-Trp by combining the amination reaction by the D-aminotransferase mutant of the present invention with the deamination reaction and the condensation reaction to carry out the deamination reaction, the condensation reaction and the amination reaction in one reactor (on-pot enzymatic reaction) (FIG. 1).

### Example 2

### Construction of DAT-Expressing Strain Derived from Bacillus proteiformans AJ3844 Strain and Analysis of Substrate Specificity

Genomic DNA from Bacillus proteiformans AJ3844 strain was prepared according to standard methods, and a DNA fragment including a DAT gene was amplified by PCR using this as a template. A sequence of the DAT gene derived from Bacillus proteiformans AJ3844 strain is as shown in SEQ ID NO:7, and those skilled in the art can synthesize an entire fragment including the DNA fragment and restriction enzyme sites required for the DNA fragment by PCR and the like. The primer Brevis-F-NdeI [5′-GGAATTCCATATGCTCTATGTAGATGGGAAATGGGTAGAAG-3′ (SEQ ID NO:9)] and the primer Brevis-F-XhoI [5′-CCCTCGAGCACGAGTACACTTGTGTTGATATGCTGTTC-3′ (SEQ ID NO:10)], and PrimeSTAR HS DNA polymerase (TaKaRa Bio) were used for PCR.

The resulting DNA fragment was treated with the restriction enzymes NdeI and XhoI, and ligated to pET-22b (Novagen) also treated with NdeI and XhoI. E. coli JM109 was transformed with this ligation solution, and an objective plasmid was selected from ampicillin resistant clones. E. coli BL21 (DE3) was transformed with this plasmid to obtain pET22-AJ3844DAT/E. coli BL21 (DE3). In this expression strain, DAT in which His-tag was added to a C-terminus is expressed. When DAT was expressed, microbial cells grown on an LB-amp (100 mg/L) agar plate were inoculated to Overnight Express Instant TB Medium (Merck) containing 100 mg/L of ampicillin, and cultured with shaking at 30° C. for 16 hours. DAT derived from AJ3844 strain was expressed under three conditions at 25° C., 30° C., and 37° C., and a D-Asp-α-KG activity was measured utilizing obtained C.F.E. (Table 2) to confirm the expression of the DAT activity.

TABLE 2
Effect of temperature on expression
of DAT derived from AJ3844 strain

| Temperature for | Activity for |
|---|---|
| Expression (° C.) | D-Asp-αKG (U/mg) |
| 25 | 49.5 |
| 30 | 67.5 |
| 37 | 50.8 |

DAT derived from AJ3844 strain was purified from the expression strain. Microbial cells of the expression strain, pET22-AJ3844DAT/E. coli BL21 (DE3) grown on the LB-amp (100 mg/L) agar plate were inoculated to 100 mL of Overnight Express Instant TB Medium (Merck) containing 100 mg/L of ampicillin, and cultured with shaking at 37° C. for 16 hours using a Sakaguchi flask. After the termination of the cultivation, microbial cells were collected from about 200 mL of the resulting cultured medium by centrifugation, and purified using a His-Bind column. The microbial cells were washed with and suspended in 20 mM Tris-HCl (pH 7.6), 300 mM NaCl and 10 mM imidazole, and disrupted by sonication. Microbial cell debris was removed from the disrupted solution by centrifugation, and the resulting supernatant was used as a soluble fraction. A purification scheme by His-tag affinity chromatography is shown below.

For a fraction containing an eluted protein, a solution obtained by dialysis against 20 mM Tris-HCl (pH 7.6), 10 μM PLP, and 300 mM KCl was used as an enzyme solution.

TABLE 3
Purification scheme by His-tag affinity
chromatography

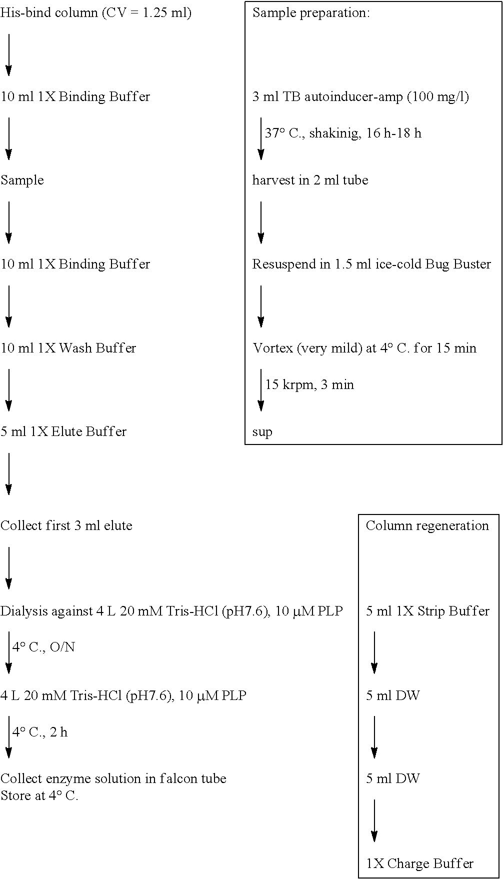

Activities for D-Asp-αKG, D-Asp-PA, D-Asp-(R)-IHOG, D-Asp-MHOG, and D-Asp-IPA were measured using the purified AJ3844 DAT solution as an enzyme source. The enzyme was diluted using 20 mM Tris-HCl (pH 7.6) and 0.01% BSA. A method for measuring each activity is shown below.

### Activity for D-Asp-αKG

A reaction was carried out in 100 mM D-Asp (pH 8.0 adjusted with NaOH), 10 mM αKG-2Na, 50 μM PLP, 100 mM Tris-HCl (pH 8.0), 0.25 mM NADH, and 2 U/mL MDH at 25° C., and the activity was calculated from the reduction of the absorbance measured at 340 nm.

### Activity for D-Asp-PA

A reaction was carried out in 100 mM D-Asp (pH 8.0 adjusted with NaOH), 10 mM PA-Na, 50 μM PLP, 100 mM Tris-HCl (pH 8.0), 0.25 mM NADH, and 2 U/mL MDH at 25° C., and the activity was calculated from the reduction of the absorbance measured at 340 nm.

### Activity for D-Asp-IPA

A reaction was carried out in 100 mM D-Asp (pH 8.0 adjusted with NaOH), 10 mM IPA, 50 μM PLP, and 100 mM Tris-HCl (pH 8.0) (pH was adjusted to pH 8.0 after preparing the reaction solution) at 25° C. for 15 minutes. The reaction was stopped by the addition of a citric acid solution (pH 4.5). A supernatant obtained by centrifuging the reaction solution after stopping the reaction was subjected to UPLC analysis.

### Activity for D-Asp-(±)-MHOG

A reaction was carried out in 100 mM D-Asp (pH 8.0 adjusted with NaOH), 10 mM (±)-MHOG, 50 μM PLP, 100 mM Tris-HCl (pH 8.0), 0.25 mM NADH, 2 U/mL MDH, and 10 U/mL LDH in 0.2 mL at 25° C., and the activity was calculated from the reduction of the absorbance measured at 340 nm.

### Activity for D-Asp-(R)-IHOG

The reaction was carried out in 100 mM D-Asp (pH 8.0 adjusted with NaOH), 10 mM (R)-IHOG, 50 μM PLP, and 100 mM Tris-HCl (pH 8.0) at 25° C. for 15 minutes. The reaction was stopped by the addition of the citric acid solution (pH 4.5). A supernatant obtained by centrifuging the reaction solution after stopping the reaction was subjected to the UPLC analysis.

Malic dehydrogenase from porcine heart (Sigma) was used as MDH.

D-Lactate dehydrogenase from Leuconostoc mesenteroides (Oriental Yeast) was used as LDH.

Conditions for the UPLC analysis are as follows.

### Column: ACQUITY UPLC HSS T3 Column, 2.1×50 mm, 1.8 μm

### (Waters)

### Injection: 5 μL

Column temperature: 40° C.
Detection wavelength: 210 nm
Flow rate: 0.5 mL/minute
Mobile phase: 20 mM KH₂PO₄/CH₃CN=96/4

The substrate specificity of DAT derived from AJ3884 strain was analyzed, and consequently its nature that an RR/Trp ratio (ratio of 2R,4R-Monatin producing activity to D-Trp (by-product) producing activity, indicator for substrate specificity) was high was confirmed (Table 4).

TABLE 4
Substrate specificity of DAT derived from AJ3884 strain

| | D-Asp-αKG | D-Asp-PA | D-Asp-MHOG | D-Asp-4R-IHOG | D-Asp-IPA | | | |
|---|---|---|---|---|---|---|---|---|
| | (U/mg) | (U/mg) | (U/mg) | (U/mg) | (U/mg) | RR/Trp | RR/Ala | RR/MHG |
| Bacillus #70 | 81.1 | 196.8 | 9.8 | 2.19 | 0.18 | 12 | 0.01 | 0.2 |

### Example 3

### In Silico Screening of Highly Selective Dat

Nucleotide sequences of various DATs shown in Table 5 were subjected to OptimumGene Codon Optimization Analysis from GenScrip, and a synthesized DAT gene sequence, a gene expression efficiency of which had been optimized in E. coli and which had been treated with NdeI and XhoI was cloned in pET-22b (Novagen) to obtain a plasmid. E. coli BL21 (DE3) was transformed with the resulting plasmid to obtain a DAT-expressing clone having a His-tag in its C terminus.

Microbial cells of the DAT-expressing strain grown on the LB-amp (100 mg/L) agar plate were inoculated to 3 mL of Overnight Express Instant TB Medium (Merck) containing 100 mg/L of ampicillin, and cultured with shaking at 37° C. for 16 hours using a test tube. Subsequently, 1 mL of the resulting cultured medium was centrifuged, and microbial cells were suspended in 1 mL of BugBuster Master Mix (Novagen). The resulting suspension was shaken at 4° C. for 15 minutes to lyse the cells and use as a cell free extract (C.F.E.). A supernatant obtained by centrifuging C.F.E. was used as a soluble fraction, and the enzyme activity for the various substrates was measured in the same manner as in Example 2 (Table 5).

TABLE 5
Substrate specificity of various DATs in in silico screening

| | | | | | D-Asp-4R- | | | |
|---|---|---|---|---|---|---|---|---|
| | | D-Asp-αKG | D-Asp-PA | D-Asp-MHOG | IHOG | D-Asp-IPA | | |
| ID No | Strain | (U/mg) | (U/mg) | (U/mg) | (U/mg) | (U/mg) | RR/Trp | RR/Ala |
| 1 | Achromobacter xylsoxidans C54 | 27.3 | 125 | 0.000 | 0.010 | 0.044 | 0.2 | 0.0001 |
| 2 | Agrobacterium radiobacter K84 | 0.03 | 0.57 | 0.003 | 0.017 | 0.011 | 1.6 | 0.03 |
| 4 | Bacillus megaterium DSM 319 | 6.63 | 17.7 | 0.02 | 0.011 | 0.152 | 0.1 | 0.0006 |
| 5 | Bhalodurans | 21.1 | 144 | 0.09 | 0.050 | 0.403 | 0.1 | 0.0003 |
| 6 | Coprococcus comes ATCC 27758 | 16.8 | 234 | 0.18 | 0.084 | 0.054 | 1.6 | 0.0004 |
| 7 | Geobacillus sp. KLS-1 | 6.13 | 32.8 | 0.09 | 0.049 | 0.036 | 1.4 | 0.001 |
| 8 | Geobacillus toebii | 11.17 | 56.6 | 0.04 | 0.021 | 0.033 | 0.6 | 0.0004 |
| 9 | ID220 | 0.16 | 0.66 | 0.01 | 0.267 | 0.166 | 1.6 | 0.4 |
| 10 | Halothiobacillus neapolitanus c2 | 4.80 | 52.0 | 0.57 | 0.044 | 0.027 | 1.6 | 0.0009 |
| 11 | ID896 | 4.58 | 42.9 | 0.03 | 0.012 | 0.027 | 0.4 | 0.0003 |
| 12 | ID892 | 0.13 | 0.73 | 0.02 | 0.054 | 0.028 | 1.9 | 0.07 |
| 13 | ID904 | 0.01 | 0.89 | 0.01 | 0.003 | 0.004 | 0.7 | 0.003 |
| 18 | Paenibacillus larvae subsp. larvae BRL-230010 | 5.73 | 25.8 | 0.02 | 0.014 | 0.758 | 0.0 | 0.0005 |
| 19 | Ruminococcaceae bacterium D16 | 11.6 | 78.6 | 0.19 | 0.413 | 0.013 | 31.9 | 0.005 |
| 20 | Robiginitalea biformata HTCC2501 | 2.01 | 17.6 | 0.01 | 0.012 | 0.004 | 3.1 | 0.0007 |
| 21 | Thiobacillus denitrificans ATCC 25259 | 0.25 | 1.06 | 0.02 | 0.004 | 0.005 | 0.7 | 0.003 |
| 24 | Rhodobacter sphaeroides ATCC 17025 | 0.57 | 0.59 | 0.01 | 0.014 | 0.032 | 0.4 | 0.02 |
| 26 | Oceanibulbus indolifex HEL-45 | 0.67 | 0.58 | 0.002 | 0.008 | 0.004 | 1.9 | 0.01 |
| 28 | Lactobacillus salivarius ATCC 11741 | 0.25 | 0.98 | 0.03 | 0.064 | 0.011 | 5.8 | 0.07 |
| 29 | ID910 | 45.7 | 228 | 0.23 | 0.237 | 0.333 | 0.7 | 0.001 |
| 30 | ID906 | 3.29 | 15.9 | 0.05 | 0.023 | 0.115 | 0.2 | 0.001 |
| 33 | ID884 | 0.26 | 130 | 0.10 | 0.770 | 0.260 | 3.0 | 0.006 |

As a result, it has been demonstrated that DAT#19 (DAT derived from Ruminococcaceae bacterium D16) has a high ratio of a 2R,4R-Monatin producing activity to a D-Trp (by-product) producing activity (hereinafter represented by an RR/Trp ratio), which is 31.9, and exhibits the second highest specific activity for 4R-IHOG (0.413 U/mg) in this in silico screening candidates.

DAT#9 has been also found, which is characterized in that the specific activity for 4R-IHOG is higher (0.267 U/mg) next to DAT#19 and the specific activity for PA and MHOG is low although the RR/Trp ratio is 1.6 that is not so high because the specific activity for IPA is also high.

Next, purified enzymes DAT9 and DAT19 were prepared. Microbial cells of the DAT expression strain grown on the LB-amp (100 mg/L) agar plate were inoculated to 100 mL of Overnight Express Instant TB Medium (Merck) containing 100 mg/L of ampicillin, and cultured with shaking at 37° C. for 16 hours using a Sakaguchi flask. After the termination of the cultivation, microbial cells were collected from the resulting cultured medium by centrifugation, washed with and suspended in 20 mM Tris-HCl (pH 7.6), 300 mM NaCl and 10 mM imidazole, and disrupted by sonication. Microbial cell debris was removed from the disrupted solution by centrifugation, and the resulting supernatant was used as a soluble fraction.

The obtained soluble fraction was applied onto a His-tag protein purification column His TALON Superflow 5 ml Cartridge (Clontech) equilibrated with 20 mM Tris-HCl (pH 7.6), 300 mM NaCl, and 10 mM imidazole, and adsorbed to a carrier. Proteins that had not been adsorbed to the carrier (unadsorbed proteins) were washed out with 20 mM Tris-HCl (pH 7.6), 300 mM NaCl, and 10 mM imidazole, and subsequently the adsorbed proteins were eluted using 20 mM Tris-HCl (pH 7.6), 300 mM NaCl, and 150 mM imidazole at a flow rate of 5 mL/minute.

For a fraction containing an eluted protein, a solution obtained by dialysis against 20 mM Tris-HCl (pH 7.6), 10 μM PLP, and 300 mM KCl was used as an enzyme solution (an amount of the medium and the number of linked TALON columns were increased as need for the purification).

The specific activity for 10 mM various keto acids (αKG, PA, IPA, (±)-MHOG and 4R-IHOG) when 100 mM D-Asp was used as an amino donor was examined using the resulting purified enzyme (Table 6).

As a result, the same nature as observed in C.F.E. was confirmed for any of DATs. That is, purified DAT#19 also had the high RR/Trp ratio that was also a target of this screening, and the purified DAT#9 also had the relatively high specific activity for 4R-IHOG that was not beyond the level of DAT#19 and the low specific activity for PA and MHOG. Comparing with the specific activity for various keto acids previously measured in BMDAT-22 that was DAT previously acquired, any DAT acquired this time exhibited the higher specific activity for 4R-IHOG. DAT#9 had the higher specific activity for IPA than BMDAT-22 but the lower specific activity for PA and MHOG. Meanwhile, DAT#19 exhibited the higher specific activity for PA and MHOG.

TABLE 6
Substrate specificity of various DAT

| | D-Asp-αKG | D-Asp-PA | D-Asp-MHOG | D-Asp-4R-IHOG | D-Asp-IPA | | | |
|---|---|---|---|---|---|---|---|---|
| | (U/mg) | (U/mg) | (U/mg) | (U/mg) | (U/mg) | RR/Trp | RR/Trp | RR/Trp |
| DAT#9 | 0.65 | 1.1 | 0.03 | 0.70 | 0.32 | 2.2 | 0.6 | 20.0 |
| DAT#19 | 12.1 | 35.0 | 10.6 | 1.06 | 0.06 | 18.4 | 0.03 | 0.1 |
| BMDAT-ID22 | 11.7 | 18.5 | 2.7 | 0.17 | 0.03 | 4.9 | 0.01 | 0.06 |

### Example 4

### Construction and Evaluation of Mutant DAT Enzymes Derived from Bacillus macerans

A mutant BMDAT expression plasmid was produced by site specific mutagenesis in accordance with protocol of QuickChange Site-Directed Mutagenesis Kit supplied from Stratagene. DNA primers (two strands in pair) designed to introduce an objective nucleotide substitution and make complementary to each strand of double-stranded DNA were synthesized (Table 7). A mutant plasmid was produced in a reaction solution composition and a PCR condition shown below using pET22b-BMDAT-22 made using pET22b vector (Novagen) having a His-tag sequence in its C terminus as a template.

TABLE 7
Primer sequences for making BMDAT mutants

| DID | Mutation | | |
|---|---|---|---|
| DID-2 | H87R | ctagaaacaggacgtgtttattttcaaa | gatttgaaaataaacacgtcctgtttct |
| | | tc | ag |
| | | (SEQ ID NO: 11) | (SEQ ID NO: 12) |
| DID-8 | N100T | gggctaattcacgtacccacgttttccc | ccgggaaaacgtgggtacgtgaattagc |
| | | gg | cc |
| | | (SEQ ID NO: 13) | (SEQ ID NO: 14) |
| DID-21 | K117R | gtattaactggaaatgtacgtgcgggtg | gcacgttcacccgcacgtacatttccag |
| | | aacgtgc | ttaatac |
| | | (SEQ ID NO: 15) | (SEQ ID NO: 16) |
| DID-22 | K117Q | gtattaactggaaatgtacaggcgggtg | gcacgttcacccgcctgtacatttccag |
| | | aacgtgc | ttaatac |
| | | (SEQ ID NO: 17) | (SEQ ID NO: 18) |
| DID-23 | I145V | ggttgcgttgtgacgttaaatctttaaa | caagtttaaagatttaacgtcacaacgc |
| | | cttg | aacc |
| | | (SEQ ID NO: 19) | (SEQ ID NO: 20) |
| DID-27 | K157R | gtgcagtattagcacgtcaagaagctgc | ctccgcagcttcttgacgtgctaatact |
| | | ggag | gcac |
| | | (SEQ ID NO: 21) | (SEQ ID NO: 22) |
| DID-28 | K157Q | gtgcagtattagcacagcaagaagctgc | ctccgcagcttcttgctgtgctaatact |
| | | ggag | gcac |
| | | (SEQ ID NO: 23) | (SEQ ID NO: 24) |
| DID-29 | K157T | gtgcagtattagcaacccaagaagctgc | ctccgcagcttcttgggttgctaatact |
| | | ggag | gcac |
| | | (SEQ ID NO: 25) | (SEQ ID NO: 26) |
| DID-40 | S240T | gatgaaatcattgtgacctctgtatcta | ctctttagatacagaggtcacaatgatt |
| | | aagag | tcatc |
| | | (SEQ ID NO: 27) | (SEQ ID NO: 28) |

DID: DAT ID

TABLE 8
Reaction solution composition

| | | |
|---|---|---|
| 10 X Reaction Buffer | 2.5 | μl |
| dsDNA (10-100 ng) | 1 | μl |
| primer (10 μM) | | |
| FW | 1 | μl |
| RV | 1 | μl |
| dNTP | 1 | μl |
| Quick Solution reagent | 0.5 | μl |
| PfuTurbo ™ DNA polymerase (2.5 U/μL) | 1 | μl |
| MQ | 17.0 | μl |
| Total | 25 | μl |

TABLE 9
PCR condition

| | | | | |
|---|---|---|---|---|
| 95° C. | 2 | min | | |
| 95° C. | 20 | sec | | |
| 60° C. | 10 | sec | {close oversize brace} | X 18 |
| 68° C. | 1 | min | | |
| 68° C. | 5 | min | | |

| | |
|---|---|
| 4° C. | Hold |

The template plasmid pET22b-BMDAT-22 was cleaved by adding 1 μL of the restriction enzyme DpnI (10 U/μL) that recognized methylated DNA and cleaved it, and treating at 37° C. for 1 to 3 hours. Competent cells XL10-Gold were transformed with the resulting reaction solution. A plasmid was recovered from the transformant, and the nucleotide sequence was determined to confirm that the objective nucleotide substitution was introduced.

A plasmid extractor PI-50 (KURABO) was used for collecting the plasmid from E. coli. BigDye Terminator v3.1 Cycle Sequencing Kit (ABI) was used for the sequencing reaction for determining the nucleotide sequence. Clean SEQ Kit (BECKMAN COULTER) was used for the purification of the sample. 3130×1 Genetic Analyzer (ABI) was used for a capillary sequencer.

E. coli JM109 (DE3) was transformed with the resulting mutant BMDAT expressing plasmid to produce a mutant BMDAT expressing strain. Microbial cells from each expression strain were inoculated to 100 mL of TB-autoinducer medium (Novagen) containing 100 μg/mL of ampicillin prepared in a 500 mL Sakaguchi flask, and cultured with reciprocal shaking at 110 rpm at 37° C. overnight (16 to 18 hours).

The resulting cultured medium was transferred to a 50 mL tube, and centrifuged at 6,000×g at 4° C. for 10 minutes to collect microbial cells. After completely removing a supernatant, the microbial cells were suspended in 8 mL of BugBuster Master Mix (Novagen). The resulting suspension was secured to a rotator, inverted and mixed at room temperature for 15 minutes, and centrifuged at 6,000×g at 4° C. for 10 minutes. A supernatant was collected in a 15 mL tube, and then filtrated using a 0.45 μm filter.

The filtrate was purified using AKTAexplorer 10S (GE Healthcare), HisTALON™ Superflow™ Cartridges (1 Column Volume=5 ml) (Clontech). The column was equilibrated with one column volume of Buffer A [20 mM Tris-HCl (pH 7.6), 300 mM NaCl, 10 mM imidazole], and then the filtrate was loaded thereto. After washing the column with two column volumes of Buffer A, proteins were eluted with two column volumes of Buffer B [20 mM Tris-HCl (pH 7.6), 300 mM NaCl, 150 mM imidazole]. An eluted fraction was collected by 1 mL. A flow rate was continuously 0.5 mL/minute. The column after the elution was washed with Buffer C [20 mM Tris-HCl (pH 7.6), 300 mM NaCl, 400 mM imidazole]. The eluted fractions corresponding to 3 mL were combined, and dialyzed using a dialysis membrane, Spectra/Por 1 Standard Grade RC Membranes (SPECTRUM) against 4 L of dialysis buffer [20 mM Tris-HCl (pH 7.6), 10 μM PLP] at 4° C. overnight. An outer solution of the dialysis was changed, subsequently the dialysis was continued for additional 2 hours at 4° C., and an inner solution of the dialysis was used as a purified enzyme solution. A protein concentration was measured using Quick Start Protein Assay Kit (BIO-RAD).

An objective activity of producing the 2R,4R-Monatin from 4R-IHOG and an activity of producing D-Trp (by-product) from IPA were measured, respectively. 100 mM D-Asp was used as an amino donor substrate in a transamination reaction, the transamination reaction for 10 mM keto acid was performed, and an amount of a produced amino acid was quantified by UPLC to calculate the specific activity. An activity of producing D-Glu from αKG that was a target substrate, an activity of producing D-Ala (by-product) using PA as the substrate, and an activity of producing MHG (by-product) using MHOG as the substrate were also measured. 100 mM D-Asp was used as the amino donor substrate in the transamination reaction, and the specific activity for 10 mM keto acid was measured by a colorimetric method.

As a result of the activity measurement, it has been found that the objective 2R,4R-Monatin/D-Trp activity ratio was enhanced in the mutants shown in Table 10. Compared with a parent enzyme, BMDAT-22, DID-28 (K157Q) exhibited 5 times higher activity of producing the 2R,4R-Monatin from αKG as the substrate, and further had a 5 times higher RR/MHG activity ratio. DID-8 (N100T) was found as a mutant in which a ratio of activity of producing the 2R,4R-Monatin/activity of producing D-Ala (by-product) (hereinafter a 2R,4R-Monatin/D-Ala activity ratio) was enhanced by 7 times. DID-8 (N100T) appears to be the mutant effective for inhibiting the production of Ala (by-product) because the activity of producing the 2R,4R-Monatin was enhanced by 3 times from 0.14 to 0.44 U/mg while the activity of producing Ala (by-product) was decreased to ½ from 35 to 16 U/mg.

TABLE 10
Substrate specificity of mutant BMDAT

| | | Activity [U/mg] (amino donor: D-Asp) | | | | | | | | |
|---|---|---|---|---|---|---|---|---|---|---|
| | | | | 4R- | IPA | PA | MHOG | | | |
| | | | | IHOG | Trp | Ala | MHG | | | |
| | | Substrate | αKG | RR- | (by- | (by- | (by- | Activity ratio | | |
| Mutant | Mutation | Product | Glu | Mona | product) | product) | product) | RR/Trp | RR/Ala | RR/MHG |

| | | | | | | | | | |
|---|---|---|---|---|---|---|---|---|---|
| Parent | | 8 | 0.14 | 0.07 | 35 | 2 | 2.1 | 0.004 | 0.07 |
| enzyme | | | | | | | | | |
| DID-2 | H87R | 2 | 0.07 | 0.02 | 4 | 1 | 4.7 | 0.018 | 0.06 |
| DID-8 | N100T | 7 | 0.44 | 0.15 | 16 | 4 | 2.9 | 0.028 | 0.10 |
| DID-21 | K117R | 7 | 0.25 | 0.06 | 24 | 6 | 4.0 | 0.011 | 0.04 |
| DID-22 | K117Q | 6 | 0.20 | 0.08 | 18 | 6 | 2.6 | 0.011 | 0.03 |
| DID-23 | I145V | 6 | 0.13 | 0.05 | 18 | 2 | 2.3 | 0.007 | 0.05 |
| DID-27 | K157R | 21 | 0.30 | 0.08 | 64 | 8 | 3.7 | 0.005 | 0.04 |
| DID-28 | K157Q | 38 | 0.68 | 0.074 | 57 | 2 | 9.2 | 0.012 | 0.31 |
| DID-29 | K157T | 32 | 0.63 | 0.16 | 38 | 5 | 4.0 | 0.017 | 0.13 |
| DID-40 | S240T | 11 | 0.15 | 0.05 | 27 | 6 | 2.8 | 0.005 | 0.02 |

### Example 5

### Examination of 2R,4R-Monatin One-Pot Reaction Using Acquired DAT

The reaction was performed using purified DAT under the following condition for 22 hours. The reaction was performed with a volume of 0.4 mL using a 1.5 mL tube. DAT was added one hour after starting the reaction. A sample was appropriately sampled, diluted with TE buffer and ultrafiltrated using an Amicon Ultra-0.5 ml centrifugal filter 10 kDa, and the filtrate was analyzed. HPLC and capillary electrophoresis were used for the analysis. In addition to DAT9 and DAT19, BMDAT-22 was evaluated as DAT.

Reaction condition: 10 mM IPA, 100 mM PA-Na, 400 mM D-Asp, 1 mM MgCl₂, 50 μM PLP, 100 mM Tris-HCl, 20 mM KPB (pH 7.6), 30 U/mL SpAld (aldolase), 1 U/mL DAT (as activity for D-Asp/4R-IHOG), 10 U/mL OAA DCase (oxaloacetic acid decarboxylase), and 100 U/mL SOD (superoxide dismutase) at 25° C. and at 140 rpm.

SpAld was prepared by the following method. A DNA fragment comprising an SpAld gene was amplified by PCR using the plasmid DNA, ptrpSpALD described in Example 5 in JP 2006-204285-A as the template. The primers SpAld-f-NdeI (5′-GGAATTCCATATGACCCAGACGCGCCTCAA-3′: SEQ ID NO:29) and SpAld-r-HindIII (5′-GCCCAAGCTTTCAGTACCCCGCCAGTTCGC-3′: SEQ ID NO:30) were used. In the aldolase gene, E. coli rare codons, 6L-ctc, 13L-ctc, 18P-ccc, 38P-ccc, 50P-ccc, 77P-ccc, 81P-ccc, and 84R-cga were changed to 6L-ctg, 13L-ctg, 18P-ccg, 38P-ccg, 50P-ccg, 77P-ccg, 81P-ccg, and 84R-cgc, respectively. When 6L was changed, the primers 6L-f (5′-ACCCAGACGCGCCTGAACGGCATCATCCG-3′: SEQ ID NO: 31) and 6L-r (5′-CGGATGATGCCGTTCAGGCGCGTCTGGGT-3′: SEQ ID NO: 32) were used. When 13 L was changed, the primers 13L-f (5′-ATCATCCGCGCTCTGGAAGCCGGCAAGCC-3′: SEQ ID NO:33) and 13L-r (5′-GGCTTGCCGGCTTCCAGAGCGCGGATGAT-3′: SEQ ID NO:34) were used. When 18P was changed, the primers 18P-f (5′-GAAGCCGGCAAGCCGGCTTTCACCTGCTT-3′: SEQ ID NO:35) and 18P-r (5′-AAGCAGGTGAAAGCCGGCTTGCCGGCTTC-3′: SEQ ID NO:36) were used. When 38P was changed, the primers 38P-f (5′-CTGACCGATGCCCCGTATGACGGCGTGGT-3′: SEQ ID NO: 37) and 38P-r (5′-ACCACGCCGTCATACGGGGCATCGGTCAG-3′: SEQ ID NO: 38) were used. When 50P was changed, the primers 50P-f (5′-ATGGAGCACAACCCGTACGATGTCGCGGC-3′: SEQ ID NO: 39) and 50p-r (5′-GCCGCGACATCGTACGGGTTGTGCTCCAT-3′: SEQ ID NO: 40) were used. When 77P, 81P, and 84R were changed, the primers 77P-81P-84R-f (5′-CGGTCGCGCCGTCGGTCACCCCGATCGCGCGCATCCCGGCCA-3′: SEQ ID NO: 41) and 77P-81P-84R-r (5′-TGGCCGGGATGCGCGCGATCGGGGTGACCGACGGCGCGACCG-3′: SEQ ID NO: 42) were used. PCR was performed using KOD-plus (Toyobo) under the following condition.
1 Cycle: 94° C. for 2 minutes
25 Cycles: 94° C. for 15 seconds
   55° C. for 15 seconds
   68° C. for 60 seconds
1 Cycle: 68° C. for 60 seconds
   4° C.

The resulting DNA fragment of about 900 bp was treated with the restriction enzymes NdeI and HindIII, and ligated to pSFN Sm_Aet (Examples 1, 6, and 12 in International Publication WO2006/075486) also treated with NdeI and HindIII. E. coli JM109 was transformed with this ligation solution, an objective plasmid was selected from ampicillin resistant clones, and this plasmid was designated as pSFN-SpAld.

One loopful of E. coli JM109/pSFN-SpAld carrying the constructed plasmid pSFN-SpAld was inoculated to 50 mL of LB liquid medium containing 100 mg/L of ampicillin, and cultured with shaking at 36° C. for 8 hours using a 500 mL Sakaguchi flask. After the termination of the cultivation, 0.0006 mL of the resulting cultured medium was added to 300 mL of a seed liquid medium (10 g of glucose, 5 g of ammonium sulfate, 1.4 g of potassium dihydrogen phosphate, 0.45 g of hydrolyzed soybeans as a nitrogen amount, 1 g of magnesium sulfate heptahydrate, 0.02 g of iron (II) sulfate heptahydrate, 0.02 g of manganese (II) sulfate pentahydrate, 1 mg of thiamin hydrochloride, 0.1 mL of Disfoam GD-113K (NOF Corporation) pH 6.3, made to one liter with water) containing 100 mg/L of ampicillin in a 1000 mL jar fermenter, and seed cultivation was started. The seed cultivation was performed at 33° C. with ventilation at 1/1 vvm with stirring at 700 rpm and controlling pH at 6.3 with ammonia until glucose was consumed. Then, 15 mL of the cultured medium obtained as above was added to 285 mL of a main liquid medium (15 g of glucose, 5 g of ammonium sulfate, 3.5 g of phosphoric acid, 0.45 g of hydrolyzed soybeans as the nitrogen amount, 1 g of magnesium sulfate heptahydrate, 0.05 g of iron (II) sulfate heptahydrate, 0.05 g of manganese (II) sulfate pentahydrate, 1 mg of thiamin hydrochloride, 0.1 mL of Disfoam GD-113K (NOF Corporation) pH 6.3, made to 0.95 L with water) containing 100 mg/L of ampicillin in a 1000 mL jar fermenter, and main cultivation was started. The main cultivation was performed at 36° C. with ventilation at 1/1 vvm, pH was controlled to 6.3 with ammonia, and stirring was controlled at 700 rpm or more so that the concentration of dissolved oxygen was 5% or more. After glucose contained in the main medium was consumed, the cultivation was continued with dropping a glucose solution at 500 g/L. Total time for cultivation was 50 hours.

Microbial cells were collected by centrifugation from 100 mL of the obtained cultured medium, washed with and suspended in 20 mM Tris-HCl (pH 7.6), and disrupted by sonication at 4° C. for 30 minutes. Microbial cell debris was removed from the disrupted solution by centrifugation, and the resulting supernatant was used as a soluble fraction.

The above soluble fraction was applied to an anion exchange chromatography column HiLoad 26/10 Q Sepharose HP (supplied from GE health Care Bioscience, CV=53 mL) equilibrated with 20 mM Tris-HCl (pH 7.6), and adsorbed to the carrier. The proteins that had not been adsorbed to the carrier (unadsorbed proteins) were washed out with 20 mM Tris-HCl (pH 7.6), and subsequently, the adsorbed proteins were eluted by linearly changing the concentration of NaCl from 0 mM to 500 mM at a flow rate of 8 mL/minute. Fractions having an aldolase activity were combined, and ammonium sulfate and Tris-HCl (pH 7.6) were added thereto at final concentrations of 1 M and 20 mM, respectively.

The resulting solution was applied to a hydrophobic chromatography column HiLoad 16/10 Phenyl Sepharose HP (supplied from GE health Care Bioscience, CV=20 mL) equilibrated with 1 M ammonium sulfate and 20 mM Tris-HCl (pH 7.6), and adsorbed to the carrier. The proteins that had not been adsorbed to the carrier were washed out with 1 M ammonium sulfate and 20 mM Tris-HCl (pH 7.6), and subsequently, the adsorbed proteins were eluted by linearly changing the concentration of ammonium sulfate from 1 M to 0 M at a flow rate of 3 mL/minute. The fractions having the aldolase activity were combined and concentrated using Amicon Ultra-15 10K (Millipore). The obtained concentrated solution was diluted with 20 mM Tris-HCl (pH 7.6), and used as an SpAld solution. The aldolase activity was measured as an aldol degradation activity using PHOG as the substrate under the following condition.

Reaction condition: 50 mM Phosphate buffer (pH 7.0), 2 mM PHOG, 0.25 mM NADH, 1 mM MgCl₂, and 16 U/mL lactate dehydrogenase at 25° C.; and an absorbance at 340 nm was measured.

OAA DCase (ODC): Oxaloacetate Decarboxylase from Pseudomonas sp. (Sigma) was used. A value described by the manufacturer was used as an enzyme amount (U).

SOD: Superoxide Dismutase from bovine liver (Sigma) was used. A value described by the manufacturer was used as an enzyme amount (U).

### HPLC Condition (Monatin, Trp, IPA, IAA and IAD)

Column: CAPCELL PAK C18 TYPE MGII, 3 μm, 4.6 mm×150 mm

### (Shiseido)

Column temperature: 40° C.

Detection wavelength: 280 nm

Flow rate: 1.0 mL/minute

Mobile phase: A: 20 mM KH₂PO₄/CH₃CN=100/5; and B: CH₃CN

TABLE 11

| Time (min) | A (%) | B (%) |
|---|---|---|
| 0 | 100 | 0 |
| 6 | 100 | 0 |
| 11 | 90 | 10 |
| 25 | 90 | 10 |
| 26 | 100 | 0 |
| 30 | 100 | 0 |

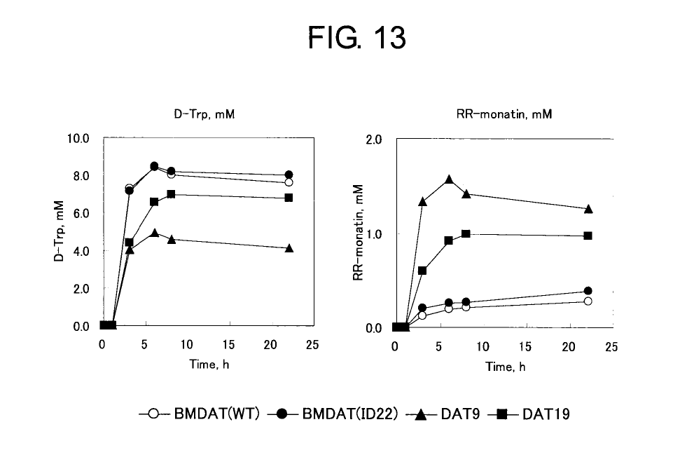

As a result of the evaluation, both DAT9 and DAT19 accumulated a higher amount of 2R,4R-Monatin than BMDAT did (FIG. 13).

### Example 6

### One-Pot Synthesis Reaction of 2R,4R-Monatin from L-Trp Using DAT9 and DAT19

A reaction using purified DAT was performed for 22 hours under the following reaction. The reaction was performed with a volume of 1 mL using a test tube. DAT was added one hour after starting the reaction. A sample was appropriately sampled, diluted with TE buffer, and ultrafiltrated using an Amicon Ultra-0.5 mL centrifugal filter 10 kDa. The resulting filtrate was analyzed. HPLC (the same condition as in 4-5-1) was used for the analysis, and L-Trp and D-Trp were quantified using HPLC using an optical resolution column.

Reaction condition: 20 mM L-Trp, 100 mM PA-Na, 400 mM D-Asp or 400 mM D-Ala, 1 mM MgCl₂, 50 μM PLP, 100 mM Tris-HCl, 20 mM KPB (pH 7.6), 5% Ps_aad broth, 30 U/mL SpAld, 1 U/mL DAT, 10 U/mL ODC (when D-Asp was added), and 100 U/mL SOD, at 25° C., and at 140 rpm.

The Ps_aad broth was prepared by the following method. One loopful of pTB2 strain that was a deaminase-expressing strain described in Example 2 in International Publication WO2009/028338 was inoculated to 50 mL of TB liquid medium containing 100 mg/L of ampicillin, and cultured with shaking at 37° C. for 16 hours using a 500 mL of Sakaguchi flask. The resulting cultured medium was used as the Ps_aad broth.

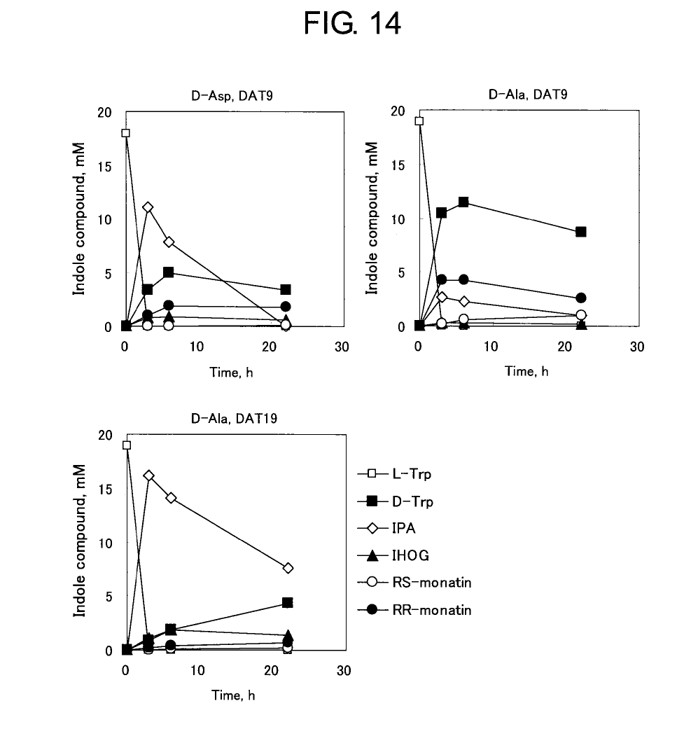

As a result, it has been confirmed that the 2R,4R-Monatin was accumulated in the both reactions and could be synthesized from L-Trp by the one-pot reaction (FIG. 14). The 2R,4R-Monatin was accumulated using DAT9 in an amount of 1.9 mM or 4.4 mM when D-Asp or D-Ala was used as the amino donor, respectively.

### Example 7

### One-Pot Synthesis Reaction of 2R,4R-Monatin Using Modified DAT

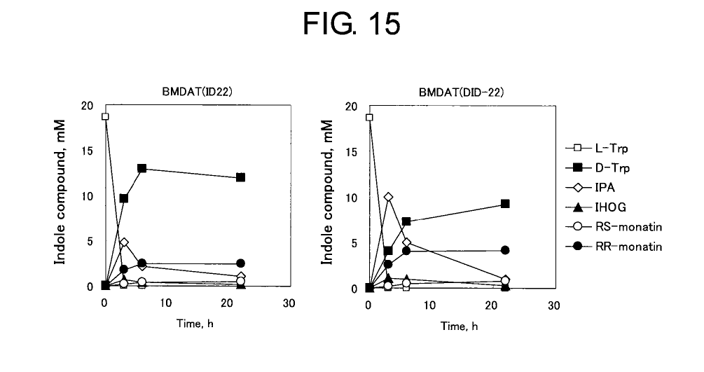

A modified enzyme BMDAT (DID-28) obtained by modifying BMDAT-22 based on its structural analysis was evaluated. According to the method described in Example 6, DID-28 was evaluated by using D-Ala as the amino donor and adding 1 U/mL of DAT one hour after starting the reaction. DID-28 exhibited the enhanced accumulation of the 2R,4R-Monatin and the reduced D-Trp (by-product) compared with ID-22 (FIG. 15).

### INDUSTRIAL APPLICABILITY

As described above, the method of the present invention is useful for producing Monatin which can be used as a sweetener.

### SEQUENCE LISTING FREE TEXT

SEQ ID NO:1: Nucleotide sequence of dat gene derived from Bacillus macerans AJ1617 (BMDAT gene)
SEQ ID NO:2: Amino acid sequence of D-aminotransferase (DAT) derived from Bacillus macerans AJ1617
SEQ ID NO:3: Forward primer for preparing D-aminotransferase mutant derived from Bacillus macerans AJ1617 (BmDAT-Nde-f)
SEQ ID NO:4: Reverse primer for preparing D-aminotransferase mutant derived from Bacillus macerans AJ1617 (BmDAT-Nde-f)
SEQ ID NO:5: Forward primer for preparing D-aminotransferase mutant derived from Bacillus macerans AJ1617 (BmDAT-delNde-f)
SEQ ID NO:6: Reverse primer for preparing D-aminotransferase mutant derived from Bacillus macerans AJ1617 (BmDAT-Xho-r)
SEQ ID NO:7: Nucleotide sequence of dat gene derived from Bacillus proteiformans AJ3844
SEQ ID NO:8: Amino acid sequence of D-aminotransferase (DAT) derived from Bacillus proteiformans AJ3844
SEQ ID NO:9: Primer for preparing D-aminotransferase derived from Bacillus proteiformans AJ3844 (Brevis-F-NdeI)
SEQ ID NO:10: Primer for preparing D-aminotransferase derived from Bacillus proteiformans AJ3844 (Brevis-F-XhoI)
SEQ ID NO:11: Forward primer for preparing D-aminotransferase mutant derived from Bacillus macerans AJ1617 (DID-2: H87R)
SEQ ID NO:12: Reverse primer for preparing D-aminotransferase mutant derived from Bacillus macerans AJ1617 (DID-2: H87R)
SEQ ID NO:13: Forward primer for preparing D-aminotransferase mutant derived from Bacillus macerans AJ1617 (DID-8: N100T)
SEQ ID NO:14: Reverse primer for preparing D-aminotransferase mutant derived from Bacillus macerans AJ1617 (DID-8: N100T)
SEQ ID NO:15: Forward primer for preparing D-aminotransferase mutant derived from Bacillus macerans AJ1617 (DID-21: K117R)
SEQ ID NO:16: Reverse primer for preparing D-aminotransferase mutant derived from Bacillus macerans AJ1617 (DID-21: K117R)
SEQ ID NO:17: Forward primer for preparing D-aminotransferase mutant derived from Bacillus macerans AJ1617 (DID-22: K117Q)
SEQ ID NO:18: Reverse primer for preparing D-aminotransferase mutant derived from Bacillus macerans AJ1617 (DID-22: K117Q)
SEQ ID NO:19: Forward primer for preparing D-aminotransferase mutant derived from Bacillus macerans AJ1617 (DID-23: I145V)
SEQ ID NO:20: Reverse primer for preparing D-aminotransferase mutant derived from Bacillus macerans AJ1617 (DID-23: I145V)
SEQ ID NO:21: Forward primer for preparing D-aminotransferase mutant derived from Bacillus macerans AJ1617 (DID-27: K157R)
SEQ ID NO:22: Reverse primer for preparing D-aminotransferase mutant derived from Bacillus macerans AJ1617 (DID-27: K157R)
SEQ ID NO:23: Forward primer for preparing D-aminotransferase mutant derived from Bacillus macerans AJ1617 (DID-28: K157Q)
SEQ ID NO:24: Reverse primer for preparing D-aminotransferase mutant derived from Bacillus macerans AJ1617 (DID-28: K157Q)
SEQ ID NO:25: Forward primer for preparing D-aminotransferase mutant derived from Bacillus macerans AJ1617 (DID-29: K157T)
SEQ ID NO:26: Reverse primer for preparing D-aminotransferase mutant derived from Bacillus macerans AJ1617 (DID-29: K157T)
SEQ ID NO:27: Forward primer for preparing D-aminotransferase mutant derived from Bacillus macerans AJ1617 (DID-40: S240T)
SEQ ID NO:28: Reverse primer for preparing D-aminotransferase mutant derived from Bacillus macerans AJ1617 (DID-40: S240T)
SEQ ID NO:29: Forward primer for amplifying DNA fragment containing SpAld gene (SpAld-f-NdeI)
SEQ ID NO:30: Reverse primer for amplifying DNA fragment containing SpAld gene (SpAld-r-HindIII)
SEQ ID NO:31: Forward primer for converting codon of aldlase gene (6L-f)
SEQ ID NO:32: Reverse primer for converting codon of aldlase gene (6L-r)
SEQ ID NO:33: Forward primer for converting codon of aldlase gene (13L-f)
SEQ ID NO:34: Reverse primer for converting codon of aldlase gene (13L-r)
SEQ ID NO:35: Forward primer for converting codon of aldlase gene (18P-f)
SEQ ID NO:36: Reverse primer for converting codon of aldlase gene (18P-r)
SEQ ID NO:37: Forward primer for converting codon of aldlase gene (38P-f)
SEQ ID NO:38: Reverse primer for converting codon of aldlase gene (38P-r)
SEQ ID NO:39: Forward primer for converting codon of aldlase gene (50P-f)
SEQ ID NO:40: Reverse primer for converting codon of aldlase gene (50P-r)
SEQ ID NO:41: Forward primer for converting codons of aldlase gene (77P-81P-84R-f)
SEQ ID NO:42: Reverse primer for converting codons of aldlase gene (77P-81P-84R-r)
SEQ ID NO:43: Polynucleotide that encodes D-aminotransferase derived from Achromobacter xylosoxidans
SEQ ID NO:44: D-aminotransferase derived from Achromobacter xylosoxidans
SEQ ID NO:45: Polynucleotide that encodes D-aminotransferase derived from Agrobacterium radiobacter
SEQ ID NO:46: D-aminotransferase derived from Agrobacterium radiobacter
SEQ ID NO:47: Polynucleotide that encodes D-aminotransferase derived from Bacillus megaterium
SEQ ID NO:48: D-aminotransferase derived from Bacillus megaterium
SEQ ID NO:49: Polynucleotide that encodes D-aminotransferase derived from B halodurans
SEQ ID NO:50: D-aminotransferase derived from B halodurans
SEQ ID NO:51: Polynucleotide that encodes D-aminotransferase derived from Coprococcus comes
SEQ ID NO:52: D-aminotransferase derived from Coprococcus comes
SEQ ID NO:53: Polynucleotide that encodes D-aminotransferase derived from Geobacillus sp.
SEQ ID NO:54: D-aminotransferase derived from Geobacillus sp.
SEQ ID NO:55: Polynucleotide that encodes D-aminotransferase derived from Geobacillus toebii
SEQ ID NO:56: D-aminotransferase derived from Geobacillus toebii
SEQ ID NO:57: Polynucleotide that encodes D-aminotransferase derived from ID220
SEQ ID NO:58: D-aminotransferase derived from ID220
SEQ ID NO:59: Polynucleotide that encodes D-aminotransferase derived from Halothiobacillus neapolitanus
SEQ ID NO:60: D-aminotransferase derived from Halothiobacillus neapolitanus
SEQ ID NO:61: Polynucleotide that encodes D-aminotransferase derived from ID896
SEQ ID NO:62: D-aminotransferase derived from ID896
SEQ ID NO:63: Polynucleotide that encodes D-aminotransferase derived from ID892
SEQ ID NO:64: D-aminotransferase derived from ID892
SEQ ID NO:65: Polynucleotide that encodes D-aminotransferase derived from ID904
SEQ ID NO:66: D-aminotransferase derived from ID904
SEQ ID NO:67: Polynucleotide that encodes D-aminotransferase derived from Paenibacillus larvae
SEQ ID NO:68: D-aminotransferase derived from Paenibacillus larvae
SEQ ID NO:69: Polynucleotide that encodes D-aminotransferase derived from Ruminococcaceae bacterium
SEQ ID NO:70: D-aminotransferase derived from Ruminococcaceae bacterium
SEQ ID NO:71: Polynucleotide that encodes D-aminotransferase derived from Robiginitalea biformata
SEQ ID NO:72: D-aminotransferase derived from Robiginitalea biformata
SEQ ID NO:73: Polynucleotide that encodes D-aminotransferase derived from Thiobacillus denitrificans
SEQ ID NO:74: D-aminotransferase derived from Thiobacillus denitrificans
SEQ ID NO:75: Polynucleotide that encodes D-aminotransferase derived from Rhodobacter sphaeroides
SEQ ID NO:76: D-aminotransferase derived from Rhodobacter sphaeroides
SEQ ID NO:77: Polynucleotide that encodes D-aminotransferase derived from Oceanibulbus indolifex
SEQ ID NO:78: D-aminotransferase derived from Oceanibulbus indolifex
SEQ ID NO:79: Polynucleotide that encodes D-aminotransferase derived from Lactobacillus salivarius
SEQ ID NO:80: D-aminotransferase derived from Lactobacillus salivarius
SEQ ID NO:81: Polynucleotide that encodes D-aminotransferase derived from ID910
SEQ ID NO:82: D-aminotransferase derived from ID910
SEQ ID NO:83: Polynucleotide that encodes D-aminotransferase derived from ID906
SEQ ID NO:84: D-aminotransferase derived from ID906
SEQ ID NO:85: Polynucleotide that encodes D-aminotransferase derived from ID884
SEQ ID NO:86: D-aminotransferase derived from ID884

1. A method for producing 2R,4R-Monatin or a salt thereof, comprising:
(1) contacting L-tryptophan with a deamination enzyme to form indole-3-pyruvate;
(2) contacting the indole-3-pyruvate and pyruvate with an aldolase to form 4R-IHOG; and
(3) contacting the 4R-IHOG with a D-aminotransferase in the presence of a D-amino acid to form the 2R,4R-Monatin.

2. The method of claim 1, wherein the steps (1)-(3) are carried out in one reactor.

3. The method of claim 1, wherein the deamination enzyme is a deaminase that is capable of acting on the L-tryptophan to form the indole-3-pyruvate.

4. The method of claim 1, wherein the D-aminotransferase has no or low ability to form D-tryptophan from the indole-3-pyruvate.

5. The method of claim 4, wherein the D-aminotransferase is derived from a microorganism belonging to genus Achromobacter, genus Agrobacterium, genus Bacillus, genus Coprococcus, genus Geobacillus, genus Halothiobacillus, genus Lactobacillus, genus Oceanibulbus, genus Paenibacillus, genus Rhodobacter, genus Robiginitalea, or genus Thiobacillus.

6. The method of claim 5, wherein the D-aminotransferase is derived from a microorganism belonging to Achromobacter xylosoxidans, Agrobacterium radiobacter, Bacillus halodurans, Bacillus megaterium, Bacillus macerans, Bacillus proteiformans, Coprococcus comes, Geobacillus sp., Geobacillus toebii, Halothiobacillus neapolitanus, Lactobacillus salivarius, Oceanibulbus indolifex, Paenibacillus larvae, Rhodobacter sphaeroides, Robiginitalea biformata, or Thiobacillus denitrificans.

7. The method of claim 4, wherein the D-aminotransferase comprises a mutation of one or more amino acid residues selected from the group consisting of the amino acid residues at positions 87, 100, 117, 145, 157, 240, 243 and 244 in the amino acid sequence represented by SEQ ID NO:2.

8. The method of claim 7, wherein the mutation of the amino acid residue is selected from the group consisting of:
i) the substitution of histidine at position 87 with arginine;
ii) the substitution of asparagine at position 100 with threonine;
iii) the substitution of lysine at position 117 with arginine or glutamine;
iv) the substitution of isoleucine at position 145 with valine;
v) the substitution of lysine at position 157 with arginine, glutamine or threonine;
vi) the substitution of serine at position 240 with threonine;
vii) the substitution of serine at position 243 with asparagine; and
viii) the substitution of serine at position 244 with lysine.

9. The method of claim 1, further comprising contacting a keto acid with a decarboxylase to degrade the keto acid, wherein the keto acid is formed from the D-amino acid due to action of the D-aminotransferase.

10. The method of claim 9, wherein the D-amino acid is D-aspartate.

11. The method of claim 10, further comprising contacting oxaloacetate with an oxaloacetate decarboxylase to irreversibly form pyruvate, wherein the oxaloacetate is formed from the D-aspartate by action of the D-aminotransferase.

12. The method of claim 11, wherein at least part of the pyruvate used in the formation of the 4R-IHOG is from pyruvate formed from the oxaloacetate due to action of the oxaloacetate decarboxylase.

13. The method of claim 1, wherein the salt is a sodium salt, a potassium salt, a magnesium salt or a calcium salt.

14. A D-aminotransferase that has an ability to form 2R,4R-Monatin from 4R-IHOG in the presence of a D-amino acid, and that has no or low ability to form D-tryptophan from indole-3-pyruvate.

15. The D-aminotransferase of claim 14, comprising a mutation of one or more amino acid residues selected from the group consisting of the amino acid residues at positions 87, 100, 117, 145, 157, 240, 243 and 244 in the amino acid sequence represented by SEQ ID NO:2.

16. The D-aminotransferase of claim 17, wherein the mutation of the amino acid residue is selected from the group consisting of:
i) the substitution of histidine at position 87 with arginine;
ii) the substitution of asparagine at position 100 with threonine;
iii) the substitution of lysine at position 117 with arginine or glutamine;
iv) the substitution of isoleucine at position 145 with valine;
v) the substitution of lysine at position 157 with arginine, glutamine or threonine;
vi) the substitution of serine at position 240 with threonine;
vii) the substitution of serine at position 243 with asparagine; and
viii) the substitution of serine at position 244 with lysine.

17. A polynucleotide encoding the D-aminotransferase of claim 14.

18. A method for producing 2R,4R-Monatin or a salt thereof, comprising the following two steps carried out in one reactor:
(1′) contacting indole-3-pyruvate and pyruvate with an aldolase to form 4R-IHOG; and
(2′) contacting the 4R-IHOG with a D-aminotransferase in the presence of a D-amino acid to form the 2R,4R-Monatin.
